SEQUENCE LISTING (8 sequences)

SEQ ID NO: 1 - DNA, 8 bp, unknown
  an Octamer example
gatccgta

SEQ ID NO: 2 - DNA, 5 bp, unknown
  an example of a sequence of nucleobases
agttc

SEQ ID NO: 3 - DNA, 5 bp, unknown
  an example of a complimentary sequence of nucleobases
gaact

SEQ ID NO: 4 - DNA, 6 bp, homo sapiens
  misc_feature 5: n=universal base
cgggnn

SEQ ID NO: 5 - DNA, 6 bp, homo sapiens
  misc_feature 2: n=universal base
nncgcc

SEQ ID NO: 6 - DNA, 17 bp, homo sapiens
acgcgggcacggctgtc

SEQ ID NO: 7 - DNA, 17 bp, homo sapiens
ctcgcggatggcgctga

SEQ ID NO: 8 - DNA, 13 bp, homo sapiens
  misc_feature 6-7: n=universal base
cggccnncgccgc

(19) United States
(12) Patent Application Publication
(10) Pub. No.: US 2005/0053979 A1
(43) Pub. Date: Mar. 10, 2005

(54) Combinatorial nucleobase oligomers comprising universal base analogues and methods for making and using same

(76) Inventors: Kenneth J. Livak, San Jose, CA (US); Khairuzzaman Bashar Mullah, Union City, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/866,523

(22) Filed: Jun. 10, 2004

Related U.S. Application Data
(60) Provisional application No. 60/478,678, filed on Jun. 12, 2003.

(51) Int. Cl. C12Q 1/68; C07H 21/04

(52) U.S. Cl. 435/6; 536/23.1

(57) ABSTRACT

The invention relates to insulating combinatorial nucleobase oligomers that comprise universal base analogs, where the oligomers are formed by the ligation of two or more oligomer “blocks” via a covalent linkage. Universal bases may serve to insulate specifically binding nucleobases from the effects of the covalent linker region joining two oligomer blocks together, so that the universal bases at least partially negate the Tₘ penalty caused by the covalent linkage, effective to reduce the required minimal length of the oligomer blocks and the combinatorial oligomer. The resulting insulating nucleobase combinatorial oligomers find use in any hybridization-based application, including use as probes and primers. The combinatorial oligomers of the present invention provide advantages over existing combinatorial oligomer systems currently known in the art.

### CROSS-REFERNCE TO RELATED APPLICATION

This application is related to, and claims the benefit under 35 U.S.C. § 119(e) of, U.S. Provisional Application Ser. No. 60/478,678, filed Jun. 12, 2003, the entire disclosure of which is hereby incorporated by reference.

### BACKGROUND OF THE INVENTION

1. Field of the Invention

This invention provides assay methods and materials pertaining to the field of synthesis of combinatorial oligomers for use in hybridizing to nucleic acids and other oligomeric molecules.

2. Introduction

Nucleic acid hybridization is a fundamental process forming the basis of a wide variety of biological research techniques and clinical applications. Hybridization-based methods are useful in the detection, quantitation and/or analysis of nucleic acids. Hybridization methods using probes and primers can include nucleic acid species such as 2′-deoxyribonucleic acid and ribonucleic acid (i.e., DNA and RNA) structures, but have been extended to alternatively incorporate non DNA or RNA structures, such as modified nucleotides and other polymeric nucleobase structures such as peptide nucleic acids (PNA) or locked nucleic acids (LNA).

Probe based assays are the basis of all studies of gene expression where selectivity for specific nucleotide species is required. Nucleic acid or other nucleobase polymer probes have long been used clinically to analyze samples for the presence of nucleic acid from bacteria, fungi, virus or other organisms, and in examining genetically-based diseases.

Nucleic acid amplification assays comprise an important class of sequence-specific detection methods used in modern biological analyses, with diverse applications in diagnosis of human disease, human identification, identification of microorganisms, paternity testing, virology, and DNA sequencing. The polymerase chain reaction (PCR) amplification method allows for the production and detection of target nucleic acid sequences with great sensitivity and specificity. PCR methods have proliferated and been adapted to form the foundation of numerous biological applications, including cloning methods, analysis of gene expression, DNA sequencing, genetic mapping, drug discovery, and numerous other applications. Methods for detecting a PCR product (i.e., an amplicon) using a nucleobase oligomer probe capable of hybridizing with the target sequence or amplicon are well known in the art.

Because the information contained in the genome and transcriptome is so large, approaches to sequence mining for genetic analysis and improving the understanding of human disease typically involve high throughput analysis that requires tens or even hundreds of thousands of probes or primers in a single application. The rapid and cost effective synthesis of nucleobase oligomers for use as probes or primers is a prerequisite for the execution of genetic analysis techniques that require large numbers (e.g., many thousands) of oligomers. Furthermore, a method for the rapid synthesis of an oligomer of defined sequence is also of more general benefit, as a request for an oligomer of defined sequence can be fulfilled and an experiment can be completed in a shorter amount of time.

Unfortunately, commercially available instruments that build custom nucleobase polymers based upon stepwise monomer assembly (de novo synthesis) require hours to produce a single oligomer, and furthermore, this production method is cost prohibitive for the manufacture of thousands of probes or primers. Therefore, it would be advantageous to have a method for the rapid, efficient and cost effective production of thousands of oligomers of desired nucleobase sequence that could be used as probes or primers for high throughput applications.

The degree of complexity of an oligomer block library is determined by the number of specificity-determining nucleobase positions in the oligomer. For example, in the case of an oligomer comprising eight specificity-determining nucleobase positions (an octamer) having the sequence:

| | |
|---|---|
| 5′-GATCCGTA-3′ | (SEQ ID NO: 1) |

can be synthesized as the ligation product of two presynthesized tetramer blocks, which would be:

5′-GATC-3′ and
5′-CGTA-3′

Assuming that each position of the presynthesized tetramer blocks can be any of the naturally occurring nucleobases, a library would have to consist of (4⁴), or 256 possible tetramer combinations. In contrast, in order to have every possible octamer presynthesized, a library of 4⁸, or 65,536 possible oligomers would need to be maintained.

Unfortunately, this approach is hindered by the attachment chemistry used to ligate the oligomer blocks. A variety of non-template-dependent linkage chemistries are widely known in the art for forming covalent linkages and ligating oligomer block subunits. However, this attachment chemistry frequently results in destabilized duplex structures when the resulting oligomers are used in hybridization reactions, e.g., when used as probes or primers. The linkages used to combine oligomer blocks introduce instability into the combinatorial oligomer. This instability is manifested in a decreased melting temperature (Tₘ) of the duplex thus formed: the Tₘ of the combinatorial oligomer is significantly lowered compared to the Tₘ an oligomer of the same base sequence that was synthesized in a single reaction without the use of oligomer blocks or linkers. This decreased Tₘ is termed a “Tₘ penalty.” The instability introduced by the chemical structure at the site of the linkage may result in a “Tₘ penalty” of as much as, for example, 10° C.

To compensate for this instability, longer (and consequently more complex and expensive) oligomers must be produced in order to achieve an acceptable dgree of hybridization stability. However, the requirement for longer oligomers also means that the complexity of the oligomer block libraries increases exponentially. For example, a tetramer oligomer library has 256 possible blocks. A pentamer library contains 1,024 possible blocks, and a hexamer library must contain 4,096 possible blocks. The maintenance of oligomer libraries significantly larger than about 6 to 8 oligomers in length becomes impractical and prohibitively expensive.

Thus, there is a need for compositions and methods for the cost-effective synthesis of combinatorial nucleobase oligomers, where the attachment chemistry used to create the oligomer does not result in the need for longer specificity-determining sequences within the oligomer to retain sufficient stability and specificity.

### SUMMARY OF THE INVENTION

The present invention provides compositions and methods which may be useful for the synthesis of combinatorial oligomers from smaller oligomer blocks linked together by a linker structure. A universal nucleobase, or a sequence of universal nucleobases can provide a spacer region that can “insulate” base-pairing segments of the oligomer block (i.e., segments comprising specificity-determining nucleobases) from the attachment linkage, where a universal nucleobase includes a base that does not significantly discriminate between bases on a complementary polymeric structure having nucleobases. A specificity-determining nucleobase is capable of discriminating between bases on a complementary polymeric structure having nucleobases. The present invention thus provides combinatorial oligomers comprising a universal nucleobase where the Tₘ penalty resulting from the linker structure is reduced or eliminated. Using the insulating combinatorial nucleobase oligomers of the present invention, it is possible to build combinatorial nucleobase oligomers in a given Tₘ range, without the need for including additional specified base sequence in order to achieve a desired Tₘ.

The present invention also provides oligomer block libraries, where collections of oligomer blocks are maintained and kept on hand for the rapid synthesis of the insulating combinatorial oligomers. An oligomer block library comprises oligomer blocks having nucleobase sequence permutations for multiple specificity-determining positions, and in addition, has at least one, and more typically more than one, universal base position in the sequence of nucleobases. An oligomer block library may include some, in some embodiments may include most, and in some embodiments may include all, possible nucleobase sequence permutations.

In an exemplary embodiment, the invention provides an insulating nucleobase oligomer block library comprising a plurality of oligomer blocks, wherein each oligomer block independently comprises polymerized nucleobases that include at least three specificity-determining nucleobases and at least one universal nucleobase, and at least one chemically reactive moiety that is covalently coupled to either or both termini of a molecule comprising polymerised nucleobases. For example, a deoxyribonucleotide oligomer block may have a chemically reactive moiety that is covalently coupled to the 3′-terminus, the 5′-terminus or both termini of the polymerized nucleobases. The chemically reactive moiety on one oligomer block is capable of reacting with the chemically reactive moiety on at least one other oligomer block to form a covalent linker between the oligomer blocks in the absence of a template to form an insulating combinatorial nucleobase oligomer. The insulating combinatorial nucleobase oligomer has a hybridization target sequence that is a composite of the specificity-determining nucleobases in the oligomer blocks comprising the insulating combinatorial nucleobase oligomer.

In some embodiments of the invention, each oligomer block independently comprises from about 3 to about 8 specificity-determining nucleobases. In some embodiments, an oligomer block library may include oligomer blocks comprising from about 1 to about 10 universal nucleobases. In some embodiments of the invention, each oligomer block independently comprises from about 1 to about 3 universal nucleobases. A universal nucleobase may be between and adjacent to two specificity-determining nucleobases. In embodiments of the invention, a universal nucleobase may be proximal to a chemically reactive moiety, distal to a chemically reactive moiety, or may be adjacent to a chemically reactive moiety.

In some embodiments, universal nucleobases comprise one or more of the following: 5-nitroindole, 3-nitropyrrole, 7-azaindole, 6-methyl-7-azaindole, pyrrollpyrizine, imidizopyridine, isocarbostyril, propynyl-7-azaindole, propynylisocarbostyril, and allenyl-7-azaindole. Universal nucleobases may also comprise one or more of the following compounds, including propynyl derivatives of the following compounds: 8-aza-7-deaza-2′-deoxyguanosine, 8-aza-7-deaza-2′-deoxyadenosine, 2′-deoxycytidine, 2′-deoxyuridine, 2′-deoxyadenosine, 2′-deoxyguanosine, and pyrrolo[2,3-d]pyrimidine nucleosides. Universal nucleobases may also comprise any of the following compounds, including derivatives therof: deoxyinosine (e.g., 2′-deoxyinosine), 7-deaza-2′-deoxyinosine, 2′-aza-2′-deoxyinosine, 3′-nitroazole, 4′-nitroindole, 5′-nitroindole, 6′-nitroindole, 4-nitrobenzimidazole, nitroindazole (e.g., 5′-nitroindazole), 4-aminobenzimidazole, imidazo-4,5-dicarboxamide, 3′-nitroimidazole, imidazole-4-carboxamide, 3-(4-nitroazol-1-yl)-1,2-propanediol, and 8-aza-7-deazaadenine (pyrazolo[3,4-d]pyrimidin-4-amine). In other examples, universal nucleobases may form universal nucleosides by combining 3-methyl-7-probynyl isocarbostyril, 3-methyl isocarbostyril, 5-methyl isocarbostyril, isocarbostyril, phenyl, or pyrenyl groups with a ribose or deoxyribose.

The invention also provides a method for the synthesis of an insulating combinatorial nucleobase oligomer, comprising selecting two or more oligomer blocks having features of the invention (e.g., selected from the oligomer block library of the invention), where the chemically reactive moieties on the oligomer blocks are capable of reacting to form a covalent linker between the oligomer blocks in the absence of a template, and reacting the selected oligomer blocks under suitable conditions whereby the chemically reactive moieties on the oligomer blocks combine to form a covalent linker between the oligomer blocks, thereby forming the insulating combinatorial nucleobase oligomer. Chemically reactive moieties suitable for the practice of the invention include carboxyl groups, aldehydes, ketones, amino groups, aminoxy groups, halides, and sulffiydryl groups, for example.

### BRIEF DESCRIPTION OF THE DRAWINGS

FIGS. 1A-1C depict generalized structures of some embodiments of the nucleobase oligomer blocks and insulating combinatorial nucleobase oligomers of the invention, where the universal bases are adjacent (i.e., proximal) to the linker. FIG. 1A shows a structure of a 5′-oligomer block; FIG. 1B shows a structure of a 3′-oligomer block; and FIG. 1C shows a structure of an insulating combinatorial nucleobase oligomer following ligation of the 5′-oligomer block and the 3′-oligomer block. Each X is a specificity-determining nucleobase, where, for example, the nucleobases can independently comprise adenine, guanine, thymine, cytosine or uracil; U is a universal nucleobase; “L” denotes the 5′ or 3′ linker chemistry; “LINKER” denotes the covalent linkage formed after ligation of the oligomer blocks; a, b, c and d are integers.

FIGS. 2A-2C depict generalized structures of some embodiments of the nucleobase oligomer blocks and insulating combinatorial nucleobase oligomers of the invention, where the universal base(s) are distal to the linker. FIG. 2A shows a structure of a 5′-oligomer block; FIG. 2B shows a structure of a 3′-oligomer block; and FIG. 2C shows a structure of an insulating combinatorial nucleobase oligomer following ligation of the 5′-oligomer block and the 3′-oligomer block. Each X is a specificity-determining nucleobase, where, for example, the nucleobases can independently comprise adenine, guanine, thymine, cytosine or uracil; U is a universal nucleobase; “L” denotes the 5′ or 3′ linker chemistry; “LINKER” denotes the covalent linkage formed after chemical ligation of the oligomer blocks; a, b, c and d are integers.

FIGS. 3A-3D depicts generalized structures of some embodiments of oligomer blocks and insulating combinatorial nucleobase oligomers of the invention, where the insulating combinatorial nucleobase oligomer is formed from the ligation of more than two oligomer blocks. FIG. 3A shows the structure of a 5′-oligomer block; FIG. 3B shows the structure of a 3′-oligomer block; FIG. 3C shows the structure of an internal oligomer block; and FIG. 3D shows a structure of an insulating combinatorial nucleobase oligomer following ligation of a 5′-oligomer block, an internal oligomer block, and a 3′-oligomer block. Each X is a specificity-determining nucleobase, where, for example, the nucleobases can independently comprise the bases adenine, guanine, thymine, cytosine or uracil; U is a universal nucleobase, “L” denotes the 5′ or 3′ linker chemistries; “LINKER” denotes the covalent linkage formed after chemical ligation of the oligomer blocks; a through g are integers.

FIGS. 4A and 4B depict two exemplary embodiments of insulating combinatorial nucleobase oligomers of the invention, where the insulating combinatorial nucleobase oligomers may or may not have contiguous complementarity with a target sequence. FIG. 4A shows an insulating combinatorial nucleobase oligomer that hybridizes to a target nucleobase sequence with contiguous complementarity (i.e., without a gap). FIG. 4B shows an insulating combinatorial nucleobase oligomer that hybridizes to a target nucleobase sequence with non-contiguous complementarity (i.e., with a gap).

FIGS. 5A and 5B depict two exemplary ligation reactions wherein one oligomer block comprises a carboxylic acid group and the second oligomer block comprises an amino group, where the two reactive groups interact to form a peptide-type bond.

FIGS. 6A-6C depict three exemplary ligation reactions involving borohydride reduction.

FIGS. 7A-7D depict exemplary ligation reactions. FIGS. 7A and 7B depict two exemplary ligation reactions involving borohydride reduction. FIG. 7C depicts an exemplary ligation reaction involving an aldehyde and an amino group. FIG. 7D depicts an exemplary Diels-Alder type ligation reaction.

FIGS. 8A-8C depict three exemplary ligation reactions involving thiol reactive groups.

FIG. 9 illustrates the structures of several non-limiting examples of universal bases. (A) 7-azaindole (labeled 7AI), (B) 6-methyl-7-azaindole (labeled M7AI), (C) pyrrollpyrizine (labeled PP), (D) imidizopyridine (labeled Impy), (E) isocarbostyril (labeled ICS), (F) propynyl-7-azaindole (labeled P7AI), (G) propynylisocarbostyril (labeled PICS), and (H) allenyl-7-azaindole (labeled A7AI). “R”, as used in this figure, is the backbone structure to which the base is attached.

FIG. 10 illustrates the structures of bases capable of base-pairing with other bases including the naturally-occurring bases. (A) 5-propynyl-Uracil; (B) 2-thio-5-propynyl-Uracil; (C) 2-thio-Thymine; (D) 2-thio-Uracil; (E) N9-(7-deaza-Guanine); (E) N9-(deaza-8-aza-Guanine); (F) N9-(2,6,-diaminopurine); (G) N8-(7-deaza-8-azaAdenine).

### DETAILED DESCRIPTION OF THE INVENTION

A. Definitions

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

The terms “nucleobase” or “base” means any nitrogen-containing heterocyclic moiety capable of forming Watson-Crick-type hydrogen bonds and stacking interactions in pairing with a complementary nucleobase or nucleobase analog (i.e., derivatives of nucleobases) when that nucleobase is incorporated into a polymeric structure. “Heterocyclic” refers to a molecule with a ring system in which one or more ring atom is a heteroatom, e.g., nitrogen, oxygen, or sulfur (i.e., not carbon).

A large number of nucleobases, nucleobase analogs and nucleobase derivatives are known. Non-limiting examples of nucleobases include purines and pyrimidines, and modified forms, e.g., 7-deazapurine. Typical nucleobases are the naturally occurring nucleobases adenine, guanine, cytosine, uracil, thymine, and analogs (Seela, U.S. Pat. No. 5,446,139) of the naturally occurring nucleobases, e.g., 7-deazaadenine, 7-deazaguanine, 7-deaza-8-azaguanine, 7-deaza-8-azaadenine, inosine, nebularine, nitropyrrole (Bergstrom, J. Amer. Chem. Soc., 117:1201-1209 [1995]), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG” (Meyer, U.S. Pat. Nos. 6,143,877 and 6,127,121; Gall, WO 01/38584), and ethenoadenine (Fasman (1989) in Practical Handbook of Biochemistry and Molecular Biology, pp. 385-394, CRC Press, Boca Raton, Fla.).

The term “nucleobase oligomer” or “oligomer” as used herein refers to a polymeric arrangement of nucleobase-containing subunits. Typically, bases may be attached to a p+olymeric backbone structure in a “nucleobase oligomer” or “oligomer.” An oligomer can be single-stranded or double-stranded, and can be complementary to the sense or antisense strand of a gene sequence, or any other nucleobase sequence. A nucleobase oligomer can hybridize with a complementary portion of a target polynucleotide to form a duplex, which can be a homoduplex or a heteroduplex. A nucleobase oligomer is typically short, for example but not exclusively, less than about 100 nucleobases in length. Linkages between nucleobase-containing subunits can be of any type. Non-limiting examples of suitable oligomeric structures include oligo 2′-deoxyribonucleotides (i.e., DNA) and oligo ribonucleotides (i.e., RNA), locked nucleic acids (LNA) and peptide nucleic acids (PNA). A nucleobase oligomer can be enzymatically extendable or enzymatically non-extendable.

As used herein, the phrase “nucleic acid” is a nucleobase polymer having a backbone formed from nucleotides, or nucleotide analogs. Preferred nucleic acids inlcude 2′-deoxyribonucleic acid and ribonucleic acid (i.e., DNA and RNA). Note that peptide nucleic acid (PNA) is a nucleic acid mimic and not a true nucleic acid.

The term “duplex” means an intermolecular or intramolecular double-stranded portion of one or more nucleobase oligomers which is base-paired through Watson-Crick, Hoogsteen, or other sequence-specific interactions of nucleobases. In one embodiment, a duplex may consist of a primer and a template strand. In another embodiment, a duplex may consist of a non-extendable nucleobase oligomer probe and a target strand. A “hybrid” means a duplex, triplex, or other base-paired complex of nucleobase oligomers interacting by base-specific interactions, i.e., Watson-Crick or Hoogsteen type interactions.

As used herein, the expressions “sequence specificity” or “sequence specific” mean the ability of two or more polymeric nucleobase sequences to hybridize by hydrogen bonding interactions that result from complementary base pairing. Non-limiting examples of standard base pairing includes adenine base pairing with thymine or uracil and guanine base pairing with cytosine. Other non-limiting examples of base-pairing motifs include, but are not limited to: adenine base pairing with any of: 5-propynyl-uracil, 2-thio-5-propynyl-uracil, 2-thiouracil or 2-thiothymine; guanine base pairing with any of: 5-methylcytosine or pseudoisocytosine; cytosine base pairing with any of: hypoxanthine, N9-(7-deaza-guanine) or N9-(7-deaza-8-aza-guanine); and thymine or uracil base pairing with any of: 2-aminopurine, N9-(2-amino-6-chloropurine) or N9-(2,6-diaminopurine).

“Nucleoside” refers to a compound consisting of a nucleobase linked to the 1′-carbon atom of a sugar, such as ribose, arabinose, xylose, and pyranose, in the natural β or the α anomeric configuration. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Ribose examples include ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides (Asseline et al., Nucl. Acids Res., 19:4067-74 [1991]), 2′-4′- and 3′-4′-linked and other “locked” or “LNA”, bicyclic sugar modifications (WO 98/22489; WO 98/39352; WO 99/14226). Exemplary LNA sugar analogs within a polynucleotide include the structures illustrated below: 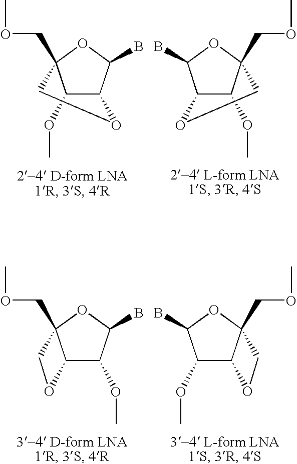
where B may be any nucleobase.

Sugars can include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi et al., Nucl. Acids Res., 21:4159-4165 (1993); Fujimori, J. Amer. Chem. Soc., 112:7435 (1990); Urata, (1993) Nucleic Acids Symposium Ser. No. 29:69-70). When the nucleobase is purine, e.g., A or G, the ribose sugar is attached to the N⁹-position of the nucleobase. When the nucleobase is pyrimidine, e.g., C, T or U, the pentose sugar is attached to the N′-position of the nucleobase.

“Nucleotide” refers to a phosphate ester of a nucleoside, as a monomer unit or within a polynucleotide polymer. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g., α-thio-nucleotide 5′-triphosphates. For a review of polynucleotide and nucleic acid chemistry, see Shabarova, Z. and Bogdanov, A. Advanced Organic Chemistry of Nucleic Acids, VCH, New York, 1994.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g., 3′-5′ and 2′-5′, inverted linkages, e.g., 3′-3′ and 5′-5′, branched structures, or internucleotide analogs. A “polynucleotide sequence” refers to the sequence of nucleotide monomers along the polymer. Polynucleotides also have associated counter ions, such as H⁺, NH⁴⁺, trialkylammonium, Mg²⁺, Na⁺ and the like.

Polynucleotides that are formed by 3′-5′ phosphodiester linkages are said to have 5′-ends and 3′-ends because the mononucleotides that are reacted to make the polynucleotide are joined in such a manner that the 5′ phosphate of one mononucleotide pentose ring is attached to the 3′ oxygen (i.e., hydroxyl) of its neighbor in one direction via the phosphodiester linkage. Thus, the 5′-end of a polynucleotide molecule has a free phosphate group or a hydroxyl at the 5′ position of the pentose ring of the nucleotide, while the 3′ end of the polynucleotide molecule has a free phosphate or hydroxyl group at the 3′ position of the pentose ring. Within a polynucleotide molecule, a position or sequence that is oriented 5′ relative to another position or sequence is said to be located “upstream,” while a position that is 3′ to another position is said to be “downstream.” This terminology reflects the fact that polymerases proceed and extend a polynucleotide chain in a 5′ to 3′ fashion along the template strand.

A polynucleotide may be composed entirely of deoxyribonucleotides, entirely of ribonucleotides, or chimeric mixtures thereof. Polynucleotides may be comprised of internucleotide, nucleobase and sugar analogs. Unless denoted otherwise, whenever a polynucleotide sequence is represented, it will be understood that the nucleotides are in 5′ to 3′ orientation from left to right and that “A” denotes deoxyadenosine, “C” denotes deoxycytidine, “G” denotes deoxyguanosine, and “T” denotes thymidine.

“Polynucleotides” are not limited to any particular length of nucleotide sequence, as the term “polynucleotides” encompasses polymeric forms of nucleotides of any length. Polynucleotides that range in size from about 5 to about 40 monomeric units are typically referred to in the art as oligonucleotides. Polynucleotides that are several thousands or more monomeric nucleotide units in length are typically referred to as nucleic acids. Polynucleotides can be linear, branched linear, or circular molecules.

Similarly, the terms “nucleobase oligomer” or “polynucleobase” describe a polymer of covalently-joined monomeric nucleobase subunits. The term does not limit the nucleobase polymer to any particular length, as these terms encompass polymeric forms of any length.

As used herein, the terms “complementary” or “complementarity” are used in reference to antiparallel strands of nucleobases (i.e., a sequence of nucleobases) related by the Watson/Crick and Hoogsteen-type base-pairing rules. For example, the sequence 5′-AGTTC-3′ (SEQ ID NO:2) is complementary to the sequence 5′-GAACT-3′. (SEQ ID NO:3).

As used herein, the term “antisense” refers to any polynucleotide or other nucleobase oligomer that is antiparallel to and complementary to another nucleobase oligomer. The term “complementary” is sometimes used interchangeably with “antisense.” The present invention encompasses antisense DNA, RNA or any other nucleobase oligomer produced by any method.

As used herein, the term “Tₘ” is used in reference to the “melting temperature.” The melting temperature is the temperature at which half of a population of double-stranded polynucloetide molecules or nucleobase oligomers, in homoduplexes or heteroduplexes, become dissociated into single strands. The Tₘ of a double-stranded nucleobase oligomeric molecule is influenced by the types of bases, the base sequence, structure of the oligomeric linkages, and the presence of non-natural features in the sequence, such as artificial linkages. Methods for calculating or experimentally determining Tm are known in the art. See, for example, Breslauer et al. Proc. Natl. Acad. Sci. USA 83: 3746-3750 (1986); Baldino et al. Methods in Enzymol. 168: 761-777 (1989); and Breslauer Methods in Enzymol. 259: 221-242 (1995).

The phrase “internucleotide analog” means a phosphate ester analog or a non-phosphate analog of a polynucleotide. Exemplary phosphate ester analogs include, but are not limited to, alkylphosphonates (e.g., C₁-C₄ alkylphosphonates), methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, and boronophosphates, C₁-C₆ alkyl-phosphotriesters, and may include associated counterions.

Non-phosphate internucleotide analogs include the family of peptide nucleic acids, commonly referred to as PNA, in which the sugar/phosphate backbone of DNA or RNA has been replaced with acyclic, achiral, and neutral polyamide linkages). PNA is a non-naturally occurring molecule, and is not known to be a substrate for any polymerase enzyme, peptidase or nuclease. Because a PNA is a polyamide, it has a C-terminus (carboxyl terminus) and an N-terminus (amino terminus). The N-terminus of the PNA oligomer is the equivalent of the 5′-phosphate of an equivalent DNA or RNA oligonucleotide, and the C-terminus is equivalent to the 3′-hydroxyl terminus. As used herein, it is intended that the term “PNA” also include related structures as known in the art, especially other peptide-based nucleic acid mimics (see, e.g., WO 96/04000).

“Substituted” as used herein refers to a molecule wherein one or more hydrogen atoms are replaced with one or more non-hydrogen atoms, functional groups or moieties. For example, an unsubstituted nitrogen is —NH₂, while a substituted nitrogen is —NHCH₃. Exemplary substituents include but are not limited to halo, e.g., fluorine and chlorine, C₁-C₈ alkyl, sulfate, sulfonate, sulfone, amino, ammonium, amido, nitrile, nitro, alkoxy (—OR where R is C₁-C₁₂ alkyl), phenoxy, aromatic, phenyl, polycyclic aromatic, heterocycle, water-solubilizing group, and linking moiety.

“Alkyl” means a saturated or unsaturated, straight-chain, branched, cyclic, or substituted hydrocarbon radical derived by the removal of one hydrogen atom from a single carbon atom of a parent alkane, alkene, or alkyne. Typical alkyl groups consist of 1-12 saturated and/or unsaturated carbons, including, but not limited to, methyl, ethyl, cyanoethyl, isopropyl, butyl, and the like.

“Alkyldiyl” means a saturated or unsaturated, branched, straight chain, cyclic, or substituted hydrocarbon radical of 1-12 carbon atoms, and having two monovalent radical centers derived by the removal of two hydrogen atoms from the same or two different carbon atoms of a parent alkane, alkene or alkyne. Typical alkyldiyl radicals include, but are not limited to, 1,2-ethyldiyl (—CH₂CH₂—), 1,3-propyldiyl (—CH₂CH₂CH₂—), 1,4-butyldiyl (—CH₂CH₂CH₂CH₂—), and the like. “Alkoxydiyl” means an alkoxyl group having two monovalent radical centers derived by the removal of a hydrogen atom from the oxygen and a second radical derived by the removal of a hydrogen atom from a carbon atom. Typical alkoxydiyl radicals include, but are not limited to, methoxydiyl (—OCH₂—) and 1,2-ethoxydiyl or ethyleneoxy (—OCH₂CH₂—). “Alkylaminodiyl” means an alkylamino group having two monovalent radical centers derived by the removal of a hydrogen atom from the nitrogen and a second radical derived by the removal of a hydrogen atom from a carbon atom. Typical alkylaminodiyl radicals include, but are not limited to —NHCH₂—, —NHCH₂CH₂—, and —NHCH₂CH₂CH₂—. “Alkylamidediyl” means an alkylamide group having two monovalent radical centers derived by the removal of a hydrogen atom from the nitrogen and a second radical derived by the removal of a hydrogen atom from a carbon atom. Typical alkylamidediyl radicals include, but are not limited to —NHC(O)CH₂—, —NHC(O)CH₂CH₂—, and —NHC(O)CH₂CH₂CH₂—.

“Aryl” means a monovalent aromatic hydrocarbon radical of 5-14 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like, including substituted aryl groups.

“Aryldiyl” means an unsaturated cyclic or polycyclic hydrocarbon radical of 5-14 carbon atoms having a conjugated resonance electron system and at least two monovalent radical centers derived by the removal of two hydrogen atoms from two different carbon atoms of a parent aryl compound, including substituted aryldiyl groups.

“Substituted alkyl”, “substituted alkyldiyl”, “substituted aryl” and “substituted aryldiyl” mean alkyl, alkyldiyl, aryl and aryldiyl respectively, in which one or more hydrogen atoms are each independently replaced with another substituent. Typical substituents include, but are not limited to, F, Cl, Br, I, R, OH, —OR, —SR, SH, NH₂, NHR, NR₂, —⁺NR₃, —N═NR₂, —CX₃, —CN, —OCN, —SCN, —NCO, —NCS, —NO, —NO₂, —N₂+, —N₃, —NHC(O)R, —C(O)R, —C(O)NR₂—S(O)₂O—, —S(O)₂R, —OS(O)₂OR, —S(O)₂NR, —S(O)R, —OP(O)(OR)₂, —P(O)(OR)₂, —P(O)(O—)₂, —P(O)(OH)₂, —C(O)R, —C(O)X, —C(S)R, —C(O)OR, —CO₂—, —C(S)OR, —C(O)SR, —C(S)SR, —C(O)NR₂, —C(S)NR₂, —C(NR)NR₂, where each R is independently —H, C₁-C₆ alkyl, C₅-C₁₄ aryl, heterocycle, or linking group. Substituents also include divalent, bridging functionality, such as diazo (—N═N—), ester, ether, ketone, phosphate, alkyldiyl, and aryldiyl groups.

As used herein, “enzymatically extendable” as it applies to a nucleobase oligomer refers to a nucleobase oligomer that is capable of serving as an enzymatic substrate for the incorporation (i.e., extension) of nucleotides complementary to a polynucleotide template by a polymerase enzyme. An enzymatically extendable nucleobase oligomer can serve as a polymerase “primer” and supports primer extension. Examples of enzymatically extendable nucleobase oligomers includes oligomers comprising 2-deoxyribose polynucleotides (DNA) and ribose polynucleotides (RNA), where the oligomers have a free ribose sugar 3′ hydroxyl group.

As used herein, “enzymatically non-extendable” as it applies to a nucleobase oligomer refers to a nucleobase oligomer that is incapable of serving as an enzymatic substrate for the incorporation (i.e., extension) of nucleotides complementary to a polynucleotide template by a polymerase enzyme. An enzymatically non-extendable nucleobase oligomer can not serve as a polymerase “primer” and can not initiate primer extension. Numerous examples of enzymatically non-extendable nucleobase oligomer structures are known in the art. These structures include, for example, any polynucleotide that: (i) is lacking a hydroxyl group on the 3′ position of the ribose sugar in the 3′ terminal nucleotide, (ii) has a modification to a sugar, nucleobase, or internucleotide linkage at or near the 3′ terminal nucleotide that blocks polymerase activity, e.g., 2′-O-methyl-ribonucleotide; or (iii) nucleobase oligomers that do not utilize a ribose sugar phosphodiester backbone in their oligmeric structure. Examples of the latter include, but are not limited to, peptide nucleic acids, termed PNAs. As used herein, the terms “non-extendable oligomer” and “blocking oligomer” are used interchangeably.

Non-extendable nucleobase oligomers can be formed by using “terminator nucleotides.” Terminator nucleotides are nucleotides that are capable of being enzymatically incorporated onto a 3′ terminus of a polynucleotide through the action of a polymerase enzyme, but cannot be further extended. Thus, a terminator nucleotide is enzymatically incorporatable, but not enzymatically extendable. Examples of terminator nucleotides include 2,3-dideoxyribonucleotides (ddNTP), 2′-deoxy, 3′-fluoro nucleotide 5′-triphosphates, and labelled forms thereof.

As used herein, “target”, “target polynucleotide”, “target nucleobase sequence,” “target sequence” and the like refer to a specific polynucleobase sequence that is the subject of hybridization with a complementary nucleobase polymer (e.g., an oligomer). The nature of the target sequence is not limiting, and can be any nucleobase polymer of any sequence, composed of, for example, DNA, RNA, substituted variants and analogs thereof, or combinations thereof. The target can be single-stranded or double-stranded. In primer extension processes, the target polynucleotide which forms a hybridization duplex with the primer may also be referred to as a “template.” A template serves as a pattern for the synthesis of a complementary polynucleotide. A target sequence for use with the present invention may be derived from any living or once living organism, including but not limited to prokaryote, eukaryote, plant, animal, and virus, as well as non-natural, synthetic and/or recombinant target sequences.

As used herein, the term “probe” refers to a nucleobase oligomer that is capable of forming a duplex structure by complementary base pairing with a sequence of a target polynucleotide, and further where the duplex so formed is detected, visualized, measured and/or quantitated. In some embodiments, the probe is fixed to a solid support, such as in column, a chip or other array format.

As used herein, the term “primer” refers to a nucleobase oligomer of defined sequence that hybridizes with a complementary portion of a target sequence and is capable of initiating the enzymatic polymerization of nucleotides (i.e., is capable of undergoing primer extension). A primer, by functional definition, is enzymatically extendable.

The term “primer extension” means the process of elongating an extendable primer that is annealed to a target in the 5′→3′ direction using a template-dependent polymerase. The extension reaction uses appropriate buffers, salts, pH, temperature, and nucleotide triphosphates, including analogs and derivatives thereof, and a template-dependent polymerase. Suitable conditions for primer extension reactions are well known in the art. The template-dependent polymerase incorporates nucleotides complementary to the template strand starting at the 3′-end of an annealed primer, to generate a complementary strand.

The terms “annealing” and “hybridization” are used interchangeably and mean the base-pairing interaction of one polynucleobase with another polynucleobase that results in formation of a duplex or other higher-ordered structure. The primary interaction is base specific, i.e., A/T and G/C, by Watson/Crick and Hoogsteen-type hydrogen bonding.

The term “solid support” refers to any solid phase material upon which an oligonucleotide is synthesized, attached or immobilized. Solid support encompasses terms such as “resin”, “solid phase”, and “support.” A solid support may be composed of organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, controlled-pore-glass (CPG), or reverse-phase silica. A solid support may include such materials as silica, organic polymers, oligosaccharides, nitrocellulose, diazocellulose, glass, polystyrene, polyvinylchloride, polypropylene, polyethylene, dextran, agar, agarose, SEPHAROSE®, SEPHADEXS SEPHACRYL®, cellulose, starch, nylon, latex beads, magnetic beads, paramagnetic beads, superparamagnetic beads, and microtitre plates.

The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. A solid support may be configured in the form of a well, depression or other container, vessel, feature or location. A plurality of solid supports may be configured in an array at various locations, addressable for robotic delivery of reagents, or by detection means including scanning by laser illumination and confocal or deflective light gathering.

As used herein, “support bound” means immobilized on or to a solid support. It is understood that immobilization can occur by any means, including for example; by covalent attachment, by electrostatic immobilization, by attachment through a ligand/ligand interaction, by contact or by depositing on the surface.

As used herein, the terms “array” or “microarray” indicate a predetermined spatial arrangement of hybridizable elements (e.g., polynucleotides) present on a solid support and/or in an arrangement of vessels. Certain array formats are referred to as a “chip” or “biochip” (M. Schena, Ed. Microarray Biochip Technology, BioTechnique Books, Eaton Publishing, Natick, Mass. [2000]). An array can comprise a low-density number of addressable locations, e.g., 2 to about 12, medium-density, e.g., about a hundred or more locations, or a high-density number, e.g., a thousand or more. Typically, the array format is a geometrically-regular shape which allows for facilitated fabrication, handling, placement, stacking, reagent introduction, detection, and storage. The array may be configured in a row and column format, with regular spacing between each location. Alternatively, the locations may be bundled, mixed, or homogeneously blended for equalized treatment or sampling. An array may comprise a plurality of addressable locations configured so that each location is spatially addressable for high-throughput handling, robotic delivery, masking, or sampling of reagents. An array can also be configured to facilitate detection or quantitation by any particular means, including but not limited to, scanning by laser illumination, confocal or deflective light gathering, and chemical luminescence. In its broadest sense, “array” formats, as recited herein, include but are not limited to, arrays (i.e., an array of a multiplicity of chips), microchips, microarrays, a microarray assembled on a single chip, or any other similar format.

The terms “in operable combination,” “in operable order,” “operably linked,” “operably joined” and similar phrases as used herein refer to functional contact between molecules, such as between an enzyme and an emzyme substrate. For example, during transcription the contact between a DNA strand and RNA polymerase forms an operable combination of the molecules, and provides an operable link between the molecules.

As used herein, the term “gene expression” refers to the process of converting genetic information encoded in the genomic nucleotide sequence on a chromosome into RNA (e.g., mRNA, rRNA, tRNA, or snRNA) through “transcription” of the gene (i.e., via the enzymatic action of an RNA polymerase).

The term “sample” as used herein is used in its broadest sense. A “sample” is typically, but not exclusively, of biological origin, and can refer to any type of material obtained from animals or plants (e.g., any fluid or tissue), cultured cells or tissues, cultures of microorganisms (prokaryotic or eukaryotic), any fraction or products produced from a living (or once living) culture or cells, or synthetically produced or in vitro sample. A sample can be unpurified or purified. A purified sample can contain principally one component, e.g., total cellular RNA, total cellular mRNA, cDNA or cRNA. In some embodiments, a sample can comprise material from a non-living source, such as synthetically produced nucleobase polymers (e.g., oligomers).

As used herein, the term “in vitro” refers to an artificial environment and to processes or reactions that occur within an artificial environment. The term “in vivo” refers to the natural environment (e.g., in an animal or in a cell) and to processes or reactions that occur within a natural environment.

As used herein, the term “DNA-dependent DNA polymerase” refers to a DNA polymerase that uses deoxyribonucleic acid (DNA) as a template for the synthesis of a complementary and antiparallel DNA strand.

As used herein, the term “DNA-dependent RNA polymerase” refers to an RNA polymerase that uses deoxyribonucleic acid (DNA) as a template for the synthesis of an RNA strand. The process mediated by a DNA-dependent RNA polymerase is commonly referred to as “transcription.” Either strand in a double-stranded DNA molecule can be used as a template for RNA synthesis, and is dependent on the sequence and orientation of the RNA-polymerase promoter operably linked to the DNA molecule.

As used herein, the term “RNA-dependent DNA polymerase” refers to a DNA polymerase that uses ribonucleic acid (RNA) as a template for the synthesis of a complementary and antiparallel DNA strand. The process of generating a DNA copy of an RNA molecule is commonly termed “reverse transcription,” and the enzyme that accomplishes that is a “reverse transcriptase.” In some cases, an enzyme that demonstrates reverse transcriptase activity also demonstrates additional activities, such as but not limited to nuclease activity (e.g., RNaseH ribonuclease activity) and DNA-dependent DNA polymerase activity.

As used herein, the term “amplification” refers generally to any process that results in an increase in the amount of a molecule. As it applies to polynucleobase molecules, amplification means the production of multiple copies of a polynucleobase molecule, or a part thereof, from one or few copies or small amounts of starting material. For example, amplification of polynucleotides can encompass a variety of chemical and enzymatic processes. The generation of multiple DNA copies from one or a few copies of a template DNA molecule during a polymerase chain reaction (PCR) is a form of amplification. Amplification is not limited to the strict duplication of the starting molecule. For example, the generation of multiple RNA molecules from a single DNA molecule during the process of transcription (e.g., in vitro transcription) is a form of amplification.

As used herein, the term “polymerase chain reaction” (PCR) refers to a well known method for amplification of a segment of a target polynucleotide in a sample, where the sample can be a single polynucleotide species, or multiple polynucleotides. Generally, the PCR process consists of introducing a molar excess of two or more extendable oligonucleotide primers to a reaction mixture comprising the desired target sequence(s), where the primers are complementary to opposite strands of the double stranded target sequence. The reaction mixture is subjected to a precise program of thermal cycling in the presence of a DNA polymerase, resulting in the amplification of the desired target sequence flanked by the DNA primers. Reverse transcriptase PCR (RT-PCR) is a PCR reaction that uses RNA template and a reverse transcriptase to first generate a DNA template molecule prior to the multiple cycles of DNA-dependent DNA polymerase primer elongation. Multiplex PCR refers to PCR reactions that produce more than one amplified product in a single reaction, typically by the inclusion of more than two primers in a single reaction. Methods for a wide variety of PCR applications are widely known in the art, and described in many sources, for example, Ausubel et al. (eds.), Current Protocols in Molecular Biology, Section 15, John Wiley & Sons, Inc., New York (1994).

As used herein, the term “polymerase extension” refers to any template-dependent polymerization of a polynucleotide by any polymerase enzyme. It is not intended that the present invention be limited to the use of any particular polymerase. A polymerase can be an RNA-dependent DNA polymerase (i.e., reverse transcriptase, e.g., Moloney murine leukemia virus [MMLV] reverse transcriptase), DNA-dependent RNA polymerase (e.g., T7 polymerase, SP6 polymerase, T3 polymerase), or a DNA-dependent DNA polymerase (e.g., Taq DNA polymerase, Bst DNA polymerase, Klenow fragment, SEQUENASE™). A polymerase may or may not be thermostable, and may or may not have 3′→5′ exonuclease activity. Polymerase extension is not limited to polymerase activity that requires a primer to initiate polymerization. For example, T7 RNA polymerase does not require the presence of a primer for polymerase initiation and extension.

As used herein, a “combinatorial nucleobase oligomer” is an oligomer of nucleobases synthesized from two or more presynthesized oligomer blocks, wherein the oligomer blocks are covalently linked by a linker.

As used herein, the term “moiety” refers to a structure that is a part of some larger structure or will become part of a larger structure. For example, a “label moiety” refers to an atom or group that serves to identify a molecule to which it is attached (e.g., a fluorescent reporter dye such as a fluorescein, a rhodamine, or a benzophenoxazine), and a “linker moiety” refers to a chemical structure that may serve to link two other chemical structures (e.g., an amino acid, an aminoalkyl carboxylic acid, an alkyl diacid, an alkyloxy diacid, or an alkyldiamine).

B. Description of Embodiments of the Invention

The present invention provides “insulated combinatorial nucleobase oligomers” or synonymously “insulating combinatorial oligomers” to overcome the technical obstacle presented by the Tₘ penalty of a combinatorial oligomer synthesized with oligomer blocks compared to an oligomer of the same base sequence synthesized without the use of oligomer blocks or linkers. Previous attempts to overcome the Tₘ penalty of a combinatorial oligomer synthesized with oligomer blocks included incorporating more oligomer units: e.g., a 10-mer oligomer with a gly-gly linkage in the center has a Tₘ that is about the same as an 8-mer without a gly-gly linkage. However, such previous methods are more complex and expensive than desired. The present invention provides improvements over such complex and expensive previous methods.

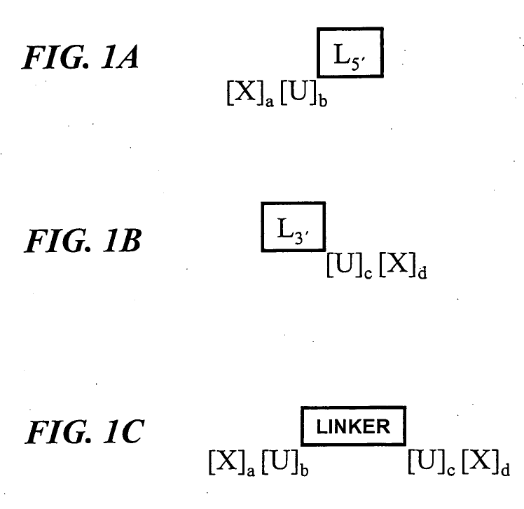

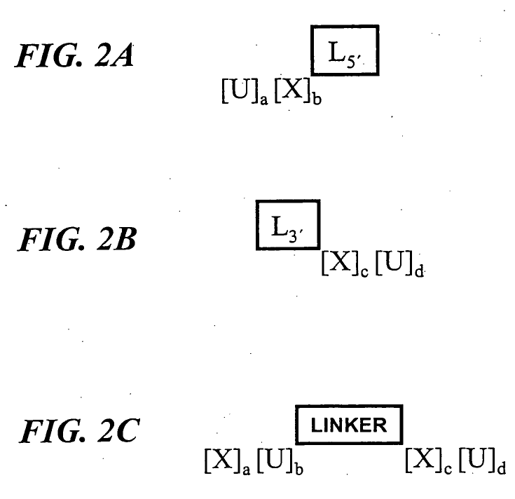

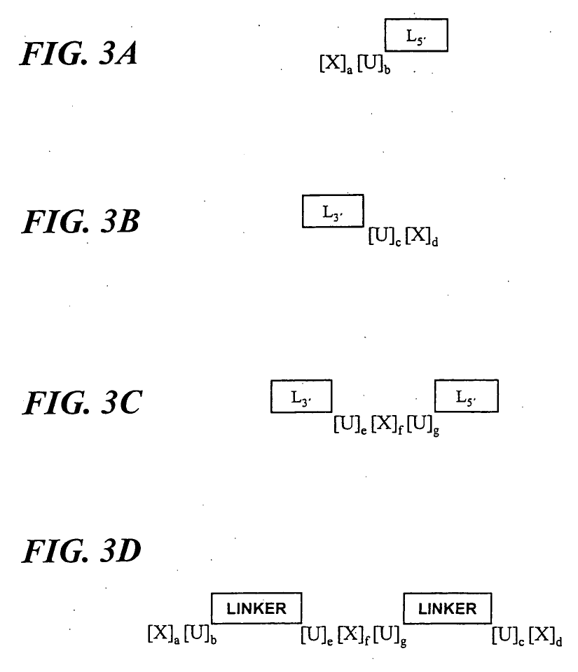

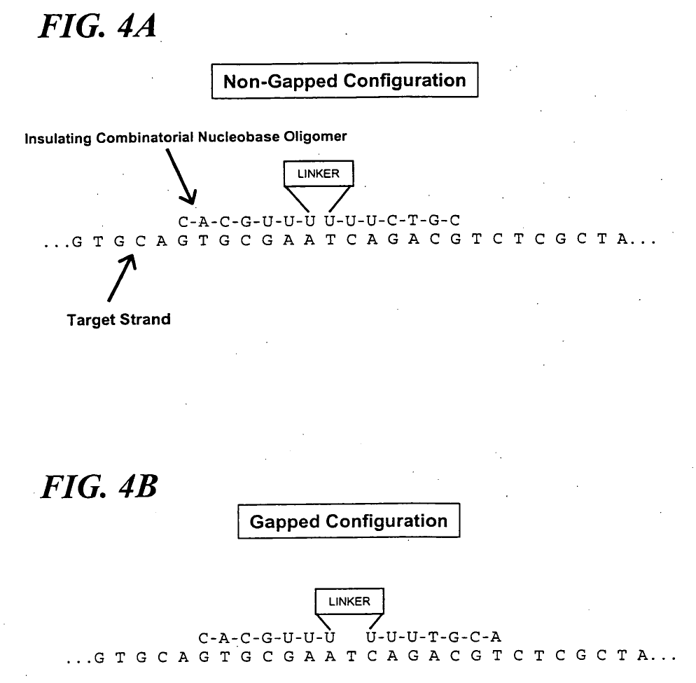

The novel insulated combinatorial nucleobase oligomers comprise two or more oligomer blocks that are covalently linked by a covalent linker, and further where the insulating combinatorial nucleobase oligomer comprises at least one universal nucleobase position. A universal nucleobase U, or a sequence of universal nucleobases can provide a spacer region that can “insulate” base-pairing segments of the oligomer block (i.e., segments comprising specificity-determining nucleobases X) from the attachment linkage, as illustrated schematically in FIGS. 1A-1C and 2A-2C. Universal bases U may be adjacent a linker, or may be separated from a linker by one or more specificity-determining nucleobases X, as illustrated in FIGS. 1A-1C and 2A-2C. An insulating combinatorial nucleobase oligomer having features of the invention may include multiple linkers, and may include multiple spacer regions, as illustrated schematically in FIGS. 3A-3D. A linker region may be of any suitable length, so that oligomer nucleobases may be contiguous, or may be separated, when the oligomer is in contact with a target nucleic acid strand, as illustrated schematically in FIGS. 4A and 4B. A universal nucleobase within the oligomer promotes hybridization stability without adding oligomer complexity, because the universal base is able to base pair with any other base in the target strand, and so does not have to be a specific base structure.

The combinatorial oligomers of this invention are referred to as insulating combinatorial nucleobase oligomers, without regard to the method of production. The hybridization properties of the oligomer result from the combined properties of the two or more component oligomer blocks and the nature of the covalent linker attaching them. The term “insulating” is used in describing the combinatorial oligomers because, in some embodiments, the universal nucleobases can be viewed as having an insulating effect to shield the specificity-determining nucleobases from any disruptive effects caused by the linker/attachment chemistry, thereby minimizing or eliminating the Tm penalty associated with many types of linker chemistries.

The insulating combinatorial oligomer can comprise nucleobases of any structure, including but not limited to, for example, polynucleotides (e.g., oligomers of 2′-deoxyribonucleotides), peptide nucleic acids (PNA), locked nucleic acids (LNA), 2′-O-alkyl oligonucleotides, 3′ modified oligodeoxyribonucleotides, N3′-P5′ phosphoramidate (NP) oligomers, MGB-oligonucleotides (minor groove binder-linked oligonucleotides), phosphorothioate (PS) oligomers, C₁-C₄ alkylphosphonate oligomers, phosphoramidates, β-phosphodiester oligonucleotides, and α-phosphodiester oligonucleotides. The insulating combinatorial oligomer of the invention is referred to as such without regard to its method of production and without regard to the linker chemistry used to ligate the oligomer blocks. The insulating combinatorial oligomers of the invention can be labeled or unlabeled. The insulating combinatorial oligomers of the invention can be used in any application comprising nucleobase oligomer hybridization, for example, as a probe or as a primer.

An aspect of the present invention concerns compositions and methods for the synthesis of combinatorial oligomers (i.e., the insulating combinatorial nucleobase oligomers) from smaller oligomer blocks, where the Tₘ penalty resulting from the linker structure is reduced or eliminated. Using the present invention, it is possible to build combinatorial oligomers in a given Tₘ range, where the oligomer does not have to include additional specified base sequence in order to achieve a desired Tₘ.

Further aspects of the present invention provide oligomer block libraries, where collections of oligomer blocks are maintained and kept on hand for the rapid synthesis of the insulating combinatorial oligomers. An oligomer block library comprises oligomer blocks having A-T-G-C sequence permutations for each specificity-determining position (or A-uracil-G-C sequence for RNA oligomers), and in addition, has at least one, and more typically more than one, universal base position in the sequence of nucleobases. For example, in the case of a tetrameric oligomer, an oligomer block library may include A-T-G-U, A-T-U-C, A-U-T-G, T-U-G-U, and other tetramers having one or more universal bases U.

In some cases, the benefits of the invention can include the advantage that smaller oligomer block libraries can be used, faster production of an oligomer with a desired sequence specificity, and lowered manufacturing costs of the oligomer, especially oligomers that will be used as probes (e.g., fluorogenic oligomers), compared with oligonucleotides that do not include features of the invention.

Insulating nucleobase oligomer blocks having features of the invention may include one, two, or more universal bases. For example, an insulating nucleobase oligomer block having features of the invention may include from about 1 to about 3, or from about 1 to about 6, or from about 1 to about 10, universal nucleobases. Insulating nucleobase oligomer blocks having features of the invention may include one, two, or three or more specificity-determining universal bases, and preferably include three or more specificity-determining bases. For example, an insulating nucleobase oligomer block having features of the invention may include from about 3 to about 8 specificity-determining bases.

An exemplary library of insulating nucleobase oligomer blocks may include multiple different insulating nucleobase oligomer blocks. An insulating nucleobase oligomer block library may include some, or most, and may, but need not, include all possible nucleobase sequence permutations. For example, an oligomer block library may include 3 different oligomer blocks, or may include 5 different oligomer blocks, or may include 10 or more different oligomer blocks, or may include 25 or more different oligomer blocks, or may include 64 or more different oligomer blocks, or may include 100 or more different oligomer blocks, or may include 200 or more different oligomer blocks, or may include 500 or more different oligomer blocks, or may include 1000 or more different oligomer blocks, or may include 5000 or more different oligomer blocks, or may include 50,000 or more different oligomer blocks, or may include 65,536 or more different oligomer blocks, or may include other numbers of different oligomer blocks. For example, a library of insulating nucleobase oligomer blocks may include at least 64 different insulating nucleobase oligomer blocks having different sequences of specificity-determining nucleobases. In some embodiments, a library of insulating nucleobase oligomer blocks may include insulating nucleobase oligomer blocks each having at least four specificity-determining nucleobases, and a library may include at least 256 different insulating nucleobase oligomer blocks having different sequences of specificity-determining nucleobases. In other embodiments, a library of insulating nucleobase oligomer blocks may include insulating nucleobase oligomer blocks each having at least five specificity-determining nucleobases, and a library may include at least 1024 different insulating nucleobase oligomer blocks having different sequences of specificity-determining nucleobases. In still further embodiments, a library of insulating nucleobase oligomer blocks may include insulating nucleobase oligomer blocks each having at least six specificity-determining nucleobases, and a library may include at least 4096 different insulating nucleobase oligomer blocks having different sequences of specificity-determining nucleobases.

According to some embodiments of the present invention, the Tm penalty caused by the oligomer block attachment chemistry can be negated or buffered by the addition of at least one, and typically more than one, universal base position that does not form base-specific interactions, but rather, has stabilizing stacking interactions with all four naturally occurring bases. In one aspect, the universal bases can be viewed as having an insulating effect if the universal bases are placed between the linker and the specificity-determining nucleobases. However, it is contemplated that the advantageous effects of incorporating universal bases are realized when the universal bases are incorporated into other positions within the combinatorial oligomer, for example, in a position distal from the linker, or interspersed between the specificity-determining bases.

It is intended that any universal base finds use with the present invention.

When using an insulating combinatorial oligomer of the invention in a hybridization reaction, the oligomer hybridizes to its target, and the universal bases pair opposite any base in the target strand. Thus, the combinatorial oligomer comprises specificity-determining bases that determine target specificity and universal bases that maintain nucleotide spacing and stacking interactions but do not contribute any base-pairing specificity with the target. The number of insulating universal spacer bases can vary depending on the type of linkage chemistry used to form the insulating combinatorial oligomer. The number of specific bases in the oligomer determines the information content of the oligomer block libraries used to make the combinatorial oligomers.

In one aspect, the specificity-determining bases of the insulating combinatorial nucleobase oligomer bind to a contiguous target sequence. In another aspect, the specificity-determining bases of the insulating combinatorial nucleobase oligomer bind to a non-contiguous (i.e., gapped) target sequence. In either case, the insulating combinatorial nucleobase oligomer complexity is the same. The fact that the blocks of specificity-determining bases interact with non-contiguous sequences in the target molecule does not change the informational complexity of the insulating combinatorial oligomer.

It is intended that the insulating combinatorial oligomers of the present invention can be synthesized using any polymeric nucleobase structure (nucleobase oligomers) that result in stable base pairing between the attached bases in the nucleobase oligomer with the bases of a target molecule. The insulating combinatorial oligomers can be enzymatically extendable, or enzymatically non-extendable. The insulating combinatorial oligomers of the invention can be synthesized from ribonucleotide triphosphates, deoxyribonucleotide triphosphates, modified nucleotides or any nucleotide analogue to yield a polymeric structure that can specifically hybridize to a target sequence. The insulating combinatorial oligomers can be synthesized from non-natural polymer backbones that contain bases yield a polymeric structure that retains the ability to specifically hybridize to a target sequence. In one embodiment, for example, PNA or LNA structures are used to form the chain of specificity-determining and universal bases in the insulating combinatorial oligomer. It is not intended that the present invention be limited to the use of any particular oligomeric structure.

In the case of using PNA oligomeric nucleobase structures and PNA oligomer block libraries, oligomer blocks with four specificity-determining bases with universal spacer bases (i.e., a tetramer library) would generate insulating combinatorial oligomers with Tₘ values as high as the combinatorial oligomers generated from libraries having five specificity-determining bases (i.e., a pentamer library) that do not contain any universal bases. The savings in manufacturing cost are due to the fact that a complete pentamer library consist of (4⁵) or 1024 oligomers, whereas, a complete tetramer library consists of only (4⁴) or 256 oligomers. Libraries of oligomer blocks with five specificity-determining bases with universal spacer bases (i.e., a pentamer library) would generate insulating combinatorial oligomers with Tₘ values as high as the combinatorial oligomers generated from libraries having six or possibly seven specificity-determining bases that do not contain any universal bases, with concomitant cost savings compared to those larger libraries not containing universal bases. Similarly, libraries of oligomer blocks with six, seven, eight, nine, ten or more specificity-determining bases with universal spacer bases would generate insulating combinatorial oligomers with Tₘ values as high as the combinatorial oligomers generated from libraries having seven, eight, nine, ten, eleven, twelve, thirteen or more specificity-determining bases that do not contain any universal bases, also with significant cost savings compared to larger libraries not containing universal bases.

In one embodiment, for example, if the number of variable specificity-determining positions in a block oligomers is 3-8, the number of block oligomers in a complete set (A, C, G and T (or uracil) as the nucleobases) is 64, 256, 1024, 4096, 16384 and 65536, respectively.

The sequence-specificity of an insulating combinatorial nucleobase oligomer of the invention is determined by the specificity-determining nucleobases in each oligomer block. The complementary sequence that can be targeted by the insulating combinatorial nucleobase oligomer of the invention is an aggregate nucleobase sequence of the oligomer blocks that are designed to hybridize to a specific target sequence of nucleobases in a sample. Accordingly, the hybridizing nucleobase sequence of the insulating combinatorial nucleobase oligomer is distributed (not necessarily evenly distributed) between at least two oligomer blocks of the insulating combinatorial nucleobase oligomer.

Consequently, with due consideration for the type of assay being used, the length and sequence requirements of a insulating combinatorial nucleobase oligomer will generally be selected such that the insulating combinatorial nucleobase oligomer will form a double stranded complex with a target sequence under suitable hybridization conditions. In one embodiment, the nucleobases of the target sequence are contiguous (i.e., there is no gap in the target sequence). In an alternative embodiment, the nucleobases of the target sequence are not contiguous (i.e., there is a gap in the target sequence). In the case where the nucleobases of the target are non-contiguous, the size of the gap is variable, and may be as few as one nucleobase, or as many as about 10 nucleobases. The type of attachment chemistry used to form the insulating combinatorial nucleobase oligomer will determine whether or not a gapped sequence should be targeted, and how large the gap should be. It is contemplated that insulating combinatorial nucleobase oligomers that utilize attachment chemistry having large, inflexible or otherwise sterically hindering chemical groups or bonds will more efficiently hybridize to a gapped target sequence.

In the simplest embodiment, for example, an insulating combinatorial oligomer of the invention is formed from the ligation of two oligomer blocks. However, the invention also provides for the ligation of more that two oligomer blocks to yield the insulating combinatorial nucleobase oligomers. In this case, the multiple oligomer blocks can be added sequentially, or combined simultaneously in a single reaction. Regardless of the method of forming a combination oligomer, it is not intended that the insulating combinatorial oligomers of the invention be limited to the covalent ligation of only two oligomer blocks. In one embodiment, the invention provides for the ligation of more than two oligomer blocks, for example, in addition to a 5′-terminal block and and a 3′-terminal oligomer block.

Regardless of the method of forming a combination oligomer as described above, in another embodiment, the product of the ligation reaction can optionally be further lengthened/elongated. Hence, the combinatorial oligomer can itself be used as an oligomer block such that repeating the method produces a further lengthened/elongated oligomer. The insulating combinatorial nucleobase oligomer, as previously formed, is reacted with a third oligomer block and optionally additional reagents under ligation conditions. This forms an elongated insulating combinatorial nucleobase oligomer. This process can be optionally repeated until the combination oligomer is of the desired length. Such a process of continued elongation can, for example, be useful for the preparation of arrays since longer oligomers are often used for this application.

Following the synthesis of the insulating combinatorial nucleobase oligomers or oligomer blocks of the invention, the oligomers may or may not be subjected to additional purification steps. Because the insulating combinatorial nucleobase oligomers are themselves produced from purified oligomer block subunits, the generation of the insulating combinatorial nucleobase oligomer results in a substantially pure reaction product, where further purification may be unnecessary. In cases where the product need not be further purified, the synthesis of the insulating combinatorial nucleobase oligomers can be rapid and cost effective once a proper block library has been constructed. Moreover, since the unreacted component oligomer blocks are typically too short to form a stable hybrid at or above ambient temperature, the unreacted component typically does not create significant problems in many types of applications.

Any conventional protocol for the purification of polymeric nucleobase structures can be used in the event that further purification of the oligomer structures of the invention is desired. Such protocols are routine in the art, and are known to one of ordinary skill, as well as described in numerous readily available sources. The degree of purity desired will determine what, if any, type of purification protocol is to be used. In one embodiment, purification is performed by a conventional method such as high performance liquid chromatography (HPLC). In other embodiments, purification is performed by any form of affinity or size exclusion chromatography. In other embodiments, purification is performed by any commercially available purification kit (using proprietary reagents).

In one aspect, the invention provides non-template directed methods for the synthesis of an insulating combinatorial nucleobase oligomer from at least two component oligomer blocks. Generally, the method comprises reacting at least two oligomer blocks (where the two oligomer blocks each comprise a suitable chemically reactive group) to provide a covalent linker that is other than the covalent bonds between the nucleobases of the 5′ and 3′-oligomer blocks, thereby forming the insulating combinatorial nucleobase oligomer. The insulating combinatorial nucleobase oligomer forms in the absence of a nucleobase template. The methods for synthesis will also comprise additional reagents such as a condensation reagent or reagents, and may or may not be conducted in aqueous solution. Various moieties on the oligomer blocks may or may not be protected by protecting groups during the ligation reaction.

C. Universal Bases

As used herein and in the art, the terms “universal base,” “universal spacer base,” “universal nucleobase,” “inert base,” “non-descriminatory base” or the like refer to a base that, when incorporated into a polymeric structure in the form of a nucleobase (e.g., a nucleotide or a PNA) does not significantly discriminate between bases on a complementary polymeric structure having nucleobases. Thus, a universal base may base-pair with a base on a complementary polymer, but does not base-pair in a significantly different way with different bases placed in a complementary position on an opposite polynucleobase strand. Alternatively, a universal base may not base-pair to a significant degree with any base on a complementary polymer. Where a first nucleotide sequence hybridizes with an at-least-partially-complementary second nucleotide squence, a universal base included in the first nucleotide sequence is effective to reduce the Tₘ penalty that would otherwise result by inclusion of a mismatched nucleotide in that position of the first nucleotide sequence. A universal base may be effective to reduce the Tₘ penalty with respect to such mismatches by about 1° C., or by about 2° C., or by about 4° C., or by about 6° C., or by about 8° C., or by about 10° C., or by greater amounts. Similarly, groups or combinations of universal bases may be effective to reduce the Tₘ penalty of sequence mismatches by about 2° C., or by about 5° C., or by about 10° C., or by about 15° C., or by about 25° C., or by about 50° C., or by greater amounts. Such reductions in the Tₘ penalty of sequence mismatches may be the result of reduced amounts of destabilization of hybridization by about 1 kcal per mole, or by about 2 kcal per mole, or by about 5 kcal per mole, or by about 15 kcal per mole, or by about 25 kcal per mole, or by about 50 kcal per mole, or by greater amounts of reduction in the energetic penalty due to sequence mismatches. A “universal nucleobase” comprises the universal base and the backbone structure.

In one embodiment, for example, a preferable universal base can pair with each of the natural bases equally well when opposite them in an oligonucleobase duplex. However, universal bases that are able to pair with a subset of the natural base also find use with the invention.

In one embodiment, for example, a universal base may be covalently attached to the 1′-carbon of a pentose sugar (e.g., ribose) backbone to make a universal nucleotide. In this case, polymerization of the nucleobases is by phosphodiester bonds to form either DNA or RNA. In another embodiment, a universal base may be covalently attached to the N-α-glycerine nitrogen of a N-[2-(aminoethyl)]glycine backbone by a methylene carbonyl linkage to a 2-aminoethylglycine polyamide polymer to make a universal PNA.

In general, universal bases are not naturally occurring and are predominantly hydrophobic molecules that can pack efficiently into duplex DNA (i.e., are able to form stacking interactions due to their hydrophobic nature). Universal bases typically refer to a nitrogen-containing aromatic heterocyclic moiety capable of participating in stable anti-parallel duplex oligomer interactions in pairing with each or some subset of the naturally occurring bases.

A universal nucleobase may or may not hydrogen bond specifically with another nucleobase. In one preferred aspect, the universal nucleobase has no preferential affinity for any particular base, but has the ability to stably base pair to any other on an antiparallel polymer of nucleobases. In other embodiments, a universal nucleobase may or may not demonstrate hydrophobic base-stacking interactions with adjacent nucleobases in a nucleobase polymer or with nucleobases in a complementary nucleobase polymer.

Thus, a universal nucleobase may be a base that does not significantly discriminate between bases on a complementary polymeric structure having nucleobases, and a specificity-determining nucleobase may be a base that is capable of discriminating between bases on a complementary polymeric structure having nucleobases.

Universal base analogues are known in the art, and include the nucleoside forms 5-nitro,1-(β-D-2-deoxyribofuranosyl)indole, termed 5-nitroindole, (see Loakes and Brown, Nucleic Acids Res. 22:4039-4043 [1994]), and 1-(2′-deoxy-β-D-ribofuranosyl)-3-nitropyrrole, termed 3-nitropyrrole (see Nichols et al., Nature 396:492-493 [1994] and Bergstrom et al., J. Am. Chem Soc. 117:1201-1209 [1995]). See also, for example, Ohtsuka et al., J. Biol. Chem. 260(5):2605-2608 (1995); Habener et al;, Proc. Natl. Acad. Sci. USA 85:1735-1739 [1988]; Van Aershot et al., Nucleic Acids Res. 23:4363-4370 [1995]; Luo et al., Nucleic Acids Res. 24:3071-3078 [1996]; Amosova et al., Nucleic Acids Res. 25:1930-1934 [1997]; Berger et al., Nucleic Acids Res. 28:2911-2914 [2000]; Seela et al., Nucleic Acids Res. 28:3224-3232 [2000]; Loakes, Nucleic Acids Res. 29:2437-2447 [2000]; Harki et al., Biochemistry 41:9026-9033 [2002]; He et al., Nucleic Acids Res. 30:5485-5496 [2002]. A universal base may be capable of forming Watson-Crick type hydrogen bonding and base stacking. Other references discussing universal bases include Berger et al., Angew. Chem. Int. Ed. Engl. (2000) 39:2940-42; Wu et al., J. Am. Chem. Soc. (2000) 122:7621-32; Berger et al., Nuc. Acids Res. (2000) 28:2911-14; Smith et al., Nucleosides & Nucleotides (1998) 17:541-554; and Ogawa et al., J. Am. Chem. Soc. (2000) 122:3274-87.

A variety of universal bases are known in the art, and include, but are not limited to: azaindole (7AI); isocarbostyril (ICS); propynylisocarbostyril (PICS); 6-methyl-7-azaindole (M7AI); imidizopyridine (ImPy); pyrrollpyrizine (PP); propynyl-7-azaindole (P7AI); and allenyl-7-azaindole (A7AI).

N8-(7-deaza-8-aza-adenine), being a universal base, base pairing with any other nucleobase, such as for example any of: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine) or N9-(7-deaza-8-aza-guanine) (See, e.g.,: Seela et al., Nucl. Acids, Res.: 28(17): 3224-3232 (2000)). Other universal bases include 5-nitroindole, 3-nitropyrrole, 6-methyl-7-azaindole, pyrrollpyrizine, imidizopyridine, isocarbostyril, propynyl-7-azaindole, propynylisocarbostyril, allenyl-7-azaindole, 8-aza-7-deaza-2′-deoxyguanosine, 8-aza-7-deaza-2′-deoxyadenosine, 2′-deoxycytidine, 2′-deoxyuridine, 2′-deoxyadenosine, 2′-deoxyguanosine, pyrrolo[2,3-d]pyrimidine, 3-nitropyrrole, deoxyinosine (e.g., 2′-deoxyinosine), 7-deaza-2′-deoxyinosine, 2′-aza-2′-deoxyinosine, 3′-nitroazole, 4′-nitroindole, 5′-nitroindole, 6′-nitroindole, 4-nitrobenzimidazole, nitroindazole (e.g., 5′-nitroindazole), 4-aminobenzimidazole, imidazo-4,5-dicarboxamide, 3′-nitroimidazole, imidazole-4-carboxamide, 3-(4-nitroazol-1-yl)-1,2-propanediol, and 8-aza-7-deazaadenine (pyrazolo[3,4-d]pyrimidin-4-amine). In other examples, universal nucleobases may form universal nucleosides by combining 3-methyl-7-propynyl isocarbostyril, 3-methyl isocarbostyril, 5-methyl isocarbostyril, isocarbostyril, phenyl, or pyrenyl groups with a ribose or deoxyribose.

D. Oligomer Blocks

As used herein, the phrase “block” or “oligomer block” describes a nucleobase oligomer comprising nucleobases functionally joined by a suitable backbone, where the oligomer block is capable of ligation with at least one additional oligomer block to generate an insulating combinatorial nucleobase oligomer. The oligomer blocks have a 5′ or 3′ orientation relative to (1) the orientation of the nucleobases and backbone in the oligomer block, and (2) relative to the oligomer block to which it will be ligated. For example, if an oligomer block has a chemical moiety attached to its 3′-terminus, that oligomer block will be ligated to an oligomer block that has suitable attachment chemistry attached to its 5′-terminus. Thus, an oligomer block can be a 5′-oligomer block or a 3′-oligomer block, for example.

The oligomer blocks may be unlabeled, labeled with one or more reporter moieties and/or comprise one or more protected or unprotected functional groups. The means by which the two oligomer blocks are ligated is not limiting, as various suitable chemistries and structures are know to one familiar with the art.

The oligomer blocks used to synthesize the insulating combinatorial nucleobase oligomers of the invention comprise minimally three parts, which are the specificity-determining nucleobases, the universal nucleobases and a chemically reactive moiety on either the 3′ terminus or 5′ terminus of the oligomer. It is not intended that the polymer backbone (i.e., the structure that serves as the scaffold for the bases) be limited to any particular chemical structure. Indeed, a wide variety of acceptable polymer structures are known in the art that find use with the invention.

In one embodiment, the nucleobase oligomers use polynucleotide chemistry to form the oligomer blocks, where the polynucleotides comprise naturally-occurring ribonucleotides, and/or 2′-deoxyribonucleotides. These structures are enzymatically extendable, and can serve as primers for the initiation of enzymatic DNA or RNA synthesis by DNA-dependent or RNA-dependent polymerases.

In other embodiments, the nucleobases used in the nucleobase oligomers are enzymatically non-extendable. That is to say, these oligomers comprise various modified nucleotide bases, nucleotide analogs or modified chain backbones that are unable to serve as primers in the initiation of enzymatic DNA or RNA synthesis by DNA-dependent or RNA-dependent polymerases. A large number of these structures are known in the art, and are described in various sources (see, e.g., WO 95/08556 and WO 99/34014). While the combinatorial nucleobase oligomer sequences of some embodiments are able to bind complementary target molecules in a sequence-specific manner, enzymatic DNA or RNA synthesis does not occur due to the non-extendable chemical structure of the nucleobase oligomer. For example, some oligomers are unable to be enzymatically extended because they lack a 3′ hydroxyl group on the ribose sugar ring required for nucleotide addition.

A large number of enzymatically non-extendable nucleobase structures are known, and find use with the present invention. It is not intended that methods of the invention be limited to the use of any one particular non-extendable nucleobase structure. Generally, enzymatically non-extendable nucleobase structures that find use with the invention may show certain advantageous properties, which may include some or all of the following: 1) oligomer blocks having defined base sequence can be readily synthesized and have some solubility in aqueous solution, 2) the resulting combinatorial oligomers are able to bind complementary target sequences in a sequence-specific manner to form stable heteroduplexes, and 3) the heteroduplexes are not subject to nuclease digestion.

It is not intended that the present invention be limited to any particular non-enzymatically extendable nucleobase oligomer structure. Examples of enzymatically non-extendable nucleobases that find use with the invention include, but are not limited to, peptide nucleic acids (PNA), locked nucleic acids (LNAs; see, WO 98/22489; WO 98/39352; and WO 99/14226), 2′-O-alkyl oligonucleotides (e.g., 2′-O-methyl modified oligonucleotides; see Majlessi et al., Nucleic Acids Research, 26(9):2224-2229 [1998]), 3′ modified oligodeoxyribonucleotides, N3′-P5′ phosphoramidate (NP) oligomers, MGB-oligonucleotides (minor groove binder-linked oligs), phosphorothioate (PS) oligomers, C₁-C₄ alkylphosphonate oligomers (e.g., methyl phosphonate (MP) oligomers), phosphoramidates, β-phosphodiester oligonucleotides, and α-phosphodiester oligonucleotides.

In addition to the modification of the termini of the oligomer blocks for ligation, the oligomer blocks can be modified and/or properly protected to thereby incorporate functional groups for labeling or for attachment to surfaces. Such functional groups can be utilized either before or after the ligation step depending upon factors such as: 1) the oligomer synthesis chemistry (e.g., harsh deprotection conditions may destroy a label), the condensation/ligation chemistry chosen (e.g., functional groups of a desired label may interfere with the condensation chemistry) and the intended use of the functional group (e.g., whether it is intended for labeling or for attachment to a solid support).

It is not intended that the bases comprising the specificity-determining nucleobase subunits be limited to the four naturally occurring bases, i.e., adenine, thymine, guanine and cytosine, or A, T, G and C, respectively. In some embodiments, non-naturally occurring bases are used in the sequence-specific nucleobase positions. The invention contemplates the use of nucleobases comprising the following non-natural bases: 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), and N9-(7-deaza-8-aza-guanine). Binding pair motifs for these bases are known in the art.

When oligomer blocks are selected for ligation to form a full length insulating combinatorial nucleobase oligomer, it is not intended that the two blocks must be of the same chemical structure or configuration. Thus, insulating combinatorial oligomers of the present invention can be chimeric in structure, for example, where the two oligomer blocks comprise nucleobases of different chemical structure (e.g., DNA and PNA). For example, see U.S. Pat. No. 6,316,230 and WO96/40709. The present invention contemplates chimeric insulating combinatorial nucleobase oligomers where oligomer blocks have different chemical structures, e.g., one oligomer block can comprise peptide nucleic acid and another oligomer block can comprise polynucleotides. These blocks can be ligated using suitable linker chemistry to form a chimeric insulating combinatorial nucleobase oligomer. It is also contemplated that the nucleobase subunits within one oligomer block can have differing chemical structure. As with uniform oligomeric structures, the chimeric oligomers of the invention may or may not be enzymatically non-extendable.

For example, the linkage of one oligomer block having D-DNA nucleotides with another oligomer block having L-DNA molecules provides a chimeric insulating combinatorial nucleobase oligomer able to be tagged by a reporter oligomer without affecting recognition of target nucleotide sequences in a sample and with minimal non-specific binding. Since L-DNA does not hybridize with D-DNA, the L-DNA-containing portion can hybridize with a complementary L-DNA oligomer having a reporter moiety without interfering with the hybridization of the D-DNA oligomer with a target nucleotide sequence in a sample, for example. The lack of binding affinity between L-DNA and D-DNA also greatly reduces non-specific binding of reporter oligomers since the L-DNA reporter oligomers will hybridize only with L-DNA, which is not found in most biological samples. Such chimeric D-DNA-L-DNA insulating combinatorial nucleobase oligomers thus can be readily labeled in the presence of target nucleotides with labels that are highly specific for the chimeric oligomers, for example.

In another aspect, the invention contemplates insulating combinatorial nucleobase oligomers that are formed by the ligation of oligomer blocks that have different configurations. For example, in one oligomer block, the universal nucleobases can be positioned adjacent to the linker chemistry, while in other configurations, the universal nucleobases can be positioned distal to the linker chemistry, or can be interspersed between the specificity-determining nucleobases. It is contemplated that oligomer blocks with different configurations, such as these, can be ligated with each other using suitable linker chemistry to form an insulating combinatorial nucleobase oligomer of the invention. It is not intended that the present invention be limited to the use of oligomer blocks that have the same chemical structure or configuration to synthesize an insulating combinatorial nucleobase oligomer.

E. Linkage Chemistry

As used herein, the term “linker” means a chemical moiety, typically at least three atoms in length, that is not part of the nucleobase-containing backbone subunit of the nucleobase polymer and provides a covalent linkage between two oligomer blocks. Prior to ligation of two oligomer blocks, atoms that will collectively form the functional covalent linker may be independently attached to the 3′ and 5′ oligomer blocks, in which case the complete linker is formed only after chemical ligation of the 3′ and 5′ blocks. Depending on its length, the linker may serve as a spacer to determine the separation between linked oligomer blocks.

As used herein, the term “ligation chemistry” is sometimes used interchangeably with the term “linker chemistry” or “attachment chemistry,” where these expressions refer to the chemical entity and reaction that will result in the functional linker that ligates the two oligomer blocks.

Prior to ligation of two oligomer blocks, atoms that will collectively form the functional covalent linker are typically independently attached to the termini of the 3′ and 5′ oligomer blocks, in which case the complete linker is formed only after functional chemical ligation of the 3′ and 5′ blocks. Linkers need not be spacers, universal nucleobases nor specificity-determining nucleobases. In embodiments of the invention, the linkers are abasic, i.e., they do not comprise a nucleobase and the atoms that define the linker are not atoms that make up the monomeric subunits of the nucleobase oligomer or any other part of the oligomer block (e.g., a spacer). In embodiments of the invention, one or more universal nucleobases may be positioned near or adjacent a linker effective to form a spacer region that may serve to insulate the specificity-determining nucleobases from the attachment linkage connecting two oligomer blocks.

It is not intended that the present invention be limited to any particular linkage chemistry or structure. On the contrary, anyone familiar with conjugation chemistry will immediately recognize a wide array of possible attachment chemistry structures that find use with the invention. Any particular chemistry or structure mentioned herein is intended to be exemplary only, and is not intended to limit the invention in any way.

It will be understood that the ligation chemistry useful in embodiments of the invention is not a limitation of the invention, so long as it produces a functional covalent linker between the oligomer blocks. A functional linker is a linker that combines the oligomer blocks in such a way that the nucleobases comprising the two (or more) oligomer blocks have the same 5′→3′ directionality, and further, the functional linker permits the specificity-determining nucleobases in the oligomer blocks to collectively determine the specificity for a complementary target sequence. That is to say, the target sequence to which the insulating combinatorial nucleobase oligomer binds is determined by the specificity-determining bases of all the oligomer blocks.

In some embodiments, for example, the act of joining the oligomer blocks is a “condensation reaction” where, in general, the ligation of the oligomer blocks results from a reaction of the chemical moieties that are attached to the termini of the oligomer blocks so as to result in the net loss of a water molecule from the reactants, in accordance with the particular chemistry chosen. The particular reaction conditions chosen will be in accordance with the particular chemistry chosen, and are well known to anyone familiar with conjugation chemistry. In some embodiments (but not all embodiments) the terms “ligation” and “condensation” are interchangeable. Strictly speaking, not all methods suitable for covalently linking (i.e., ligating) the oligomer blocks are condensation reactions.

One familiar with the art will recognize the wide range of attachment/ligation chemistries and reagents suitable for forming the insulating combinatorial oligomers of the invention. Such information is not only familiar to one of ordinary skill in the art, but is also taught in a variety of sources, including, e.g., Hermanson, Bioconjugate Techniques, Academic Press (1996).

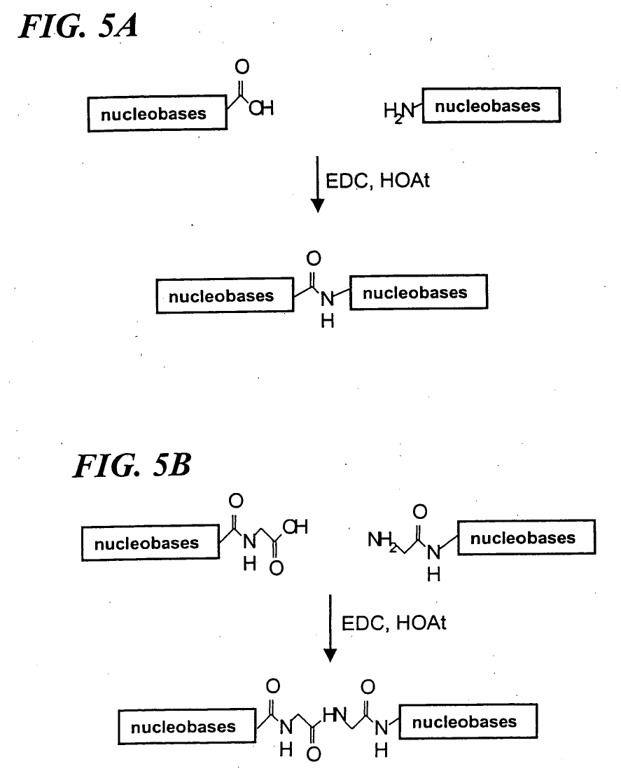

FIGS. 5-8 show several non-limiting examples of various ligation chemistries that can be used. These cited examples are intended to illustrate different types of chemistries, and it is not intended that the invention be limited in any way to any particular types of ligation chemistries.

With reference to FIGS. 5A and 5B, properly prepared oligomer blocks can be ligated using a carbodiimide, such as the water-soluble carbodiimide 1-ethyl-3-(3-dimethylamino-propyl)carbodiimide hydrochloride (EDC). As illustrated in FIG. 5A, when using this reagent, typically one of the oligomer blocks comprises a carboxylic acid moiety and the other comprises an amine group. The oligomers can be ligated in an aqueous solution, optionally containing 1 percent to 75 percent organic modifier (v/v). The pH can be less than 6.5, for example. The addition of an activating reagent such as a triazole compound (e.g., 1-hydroxy-7-azabenzotriazole [HOAt] or 1-hydroxybenzotriazole [HOBt]) may be used to increase the overall yield of the condensation/ligation reaction. Accordingly it is recommended that an activation reagent be used with the carbodiimide to effect the ligation when this chemistry is chosen.

FIG. 5A differs from FIG. 5B in that the oligomer blocks shown in FIG. 5B further comprise N-[2-aminoethyl]-glycine spacer moieties.

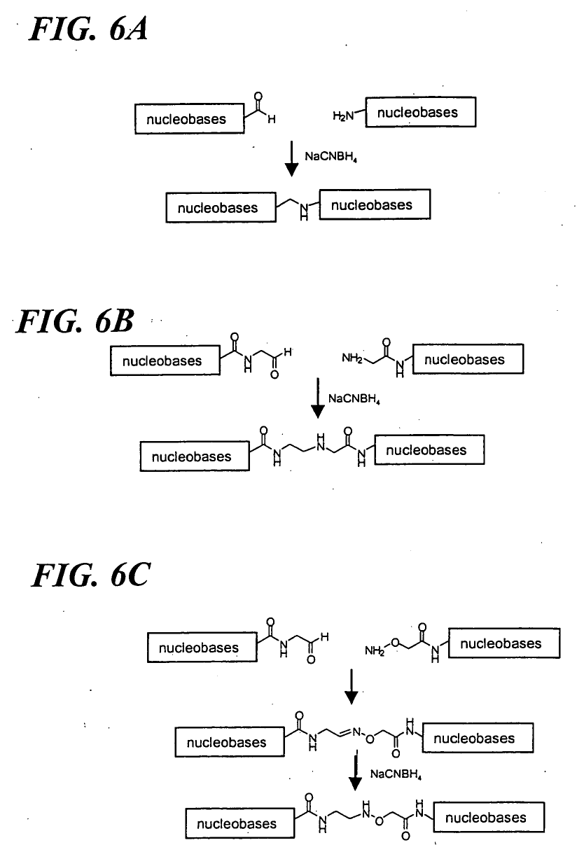

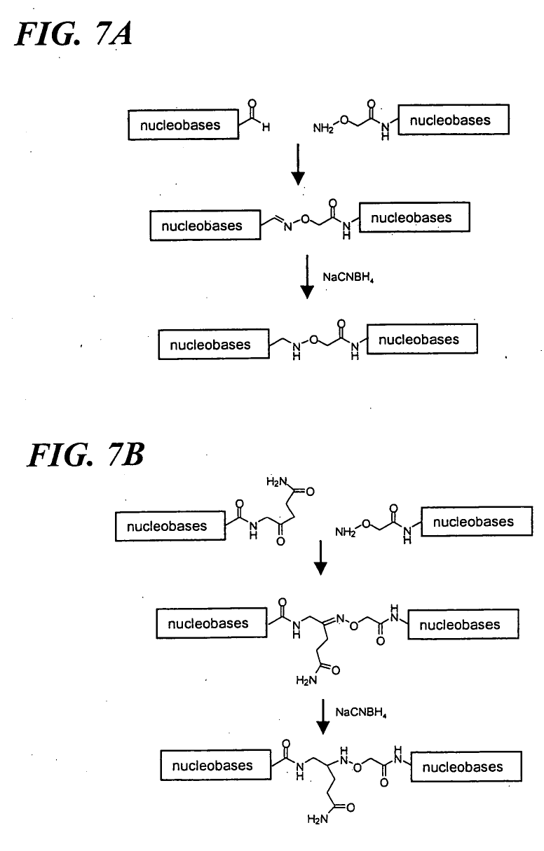

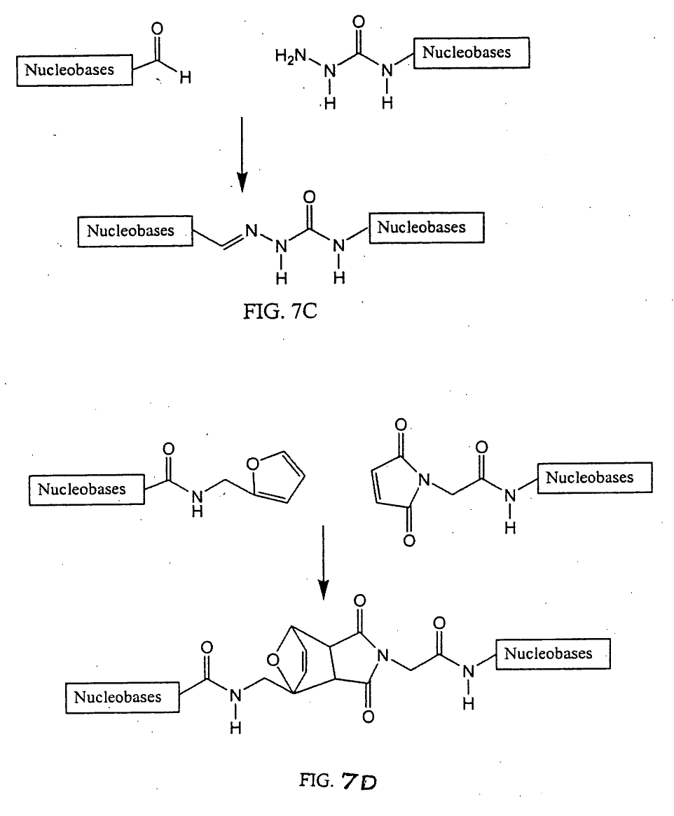

With reference to FIGS. 6A-6C and 7A and 7B, several options for the ligation/condensation of oligomer blocks are illustrated wherein sodium cyanoborohydride (NaCNBH₄) is used as a reducing reagent. It is to be understood that sodium cyanoborohydride is one of many reducing reagents that could be used to effect the ligation of the oligomer blocks using these strategies for ligation.

With reference to FIGS. 6A and 6B, one of the oligomer blocks to be ligated comprises an amine and the other oligomer block to be ligated comprises an aldehyde. The oligomer blocks can be brought into contact to thereby form an imine. Because imine formation is frequently unstable and reversible, the imine is often reduced, by for example sodium cyanoborohydride, to thereby form the ligated insulating combinatorial nucleobase oligomer. The FIGS. 6A and 6B are analogous to FIGS. 5A and 5B, where the oligomer blocks may optionally further comprise spacer moieties.

With reference to FIGS. 6C and 7A and 7B, one of the oligomer blocks to be ligated is an aldehyde or ketone, such as glycinal or β-alinal, and the other oligomer block to be ligated comprises an aminooxy-containing moiety such as aminooxyacetyl. Reaction of properly modified oligomer blocks results in the formation of an iminoxy combination oligomer that is more stable than an imine. Accordingly, the iminoxy combination oligomer can be used as prepared or can optionally be reduced with, for example, sodium cyanoborohydride to thereby form a more stable insulating combinatorial nucleobase oligomer comprising a spacer within each oligomer block, as illustrated.

FIG. 7C depicts a ligation reaction between insulating nucleobase oligomer blocks involving an aldehyde and the amino group of a semicarbazine derivative. Reaction of the chemically reactive moiety of the insulating nucleobase oligomer block depicted on the left (an aldehyde) with the chemically reactive moiety of the insulating nucleobase oligomer block depicted on the right (an amine) results in a semicarbazone forming a covalent linker as shown. The covalent linker joins the two insulating nucelobase oligomer blocks to form an insulating combinatorial nucleobase oligomer.

FIG. 7D depicts a Diels-Alder type ligation reaction to form a covalent linker joining insulating nucleobase oligomer blocks. As illustrated, a Diels-Alder type reaction between the chemically reactive moiety shown on the left (a furan derivative) and the maleimide chemically reactive moiety shown on the right to form a covalent linker that joins the insulating nucleobase oligomer blocks together into an insulating combinatorial nucleobase oligomer.

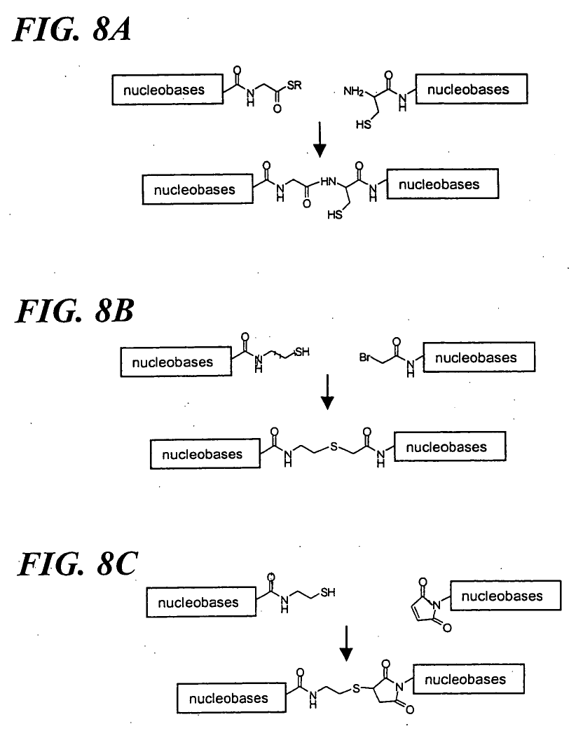

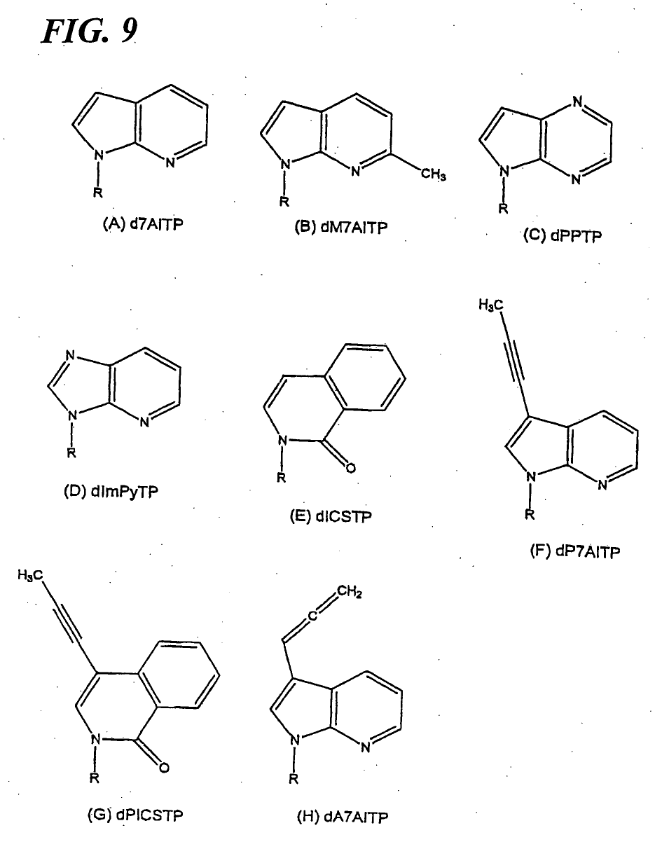

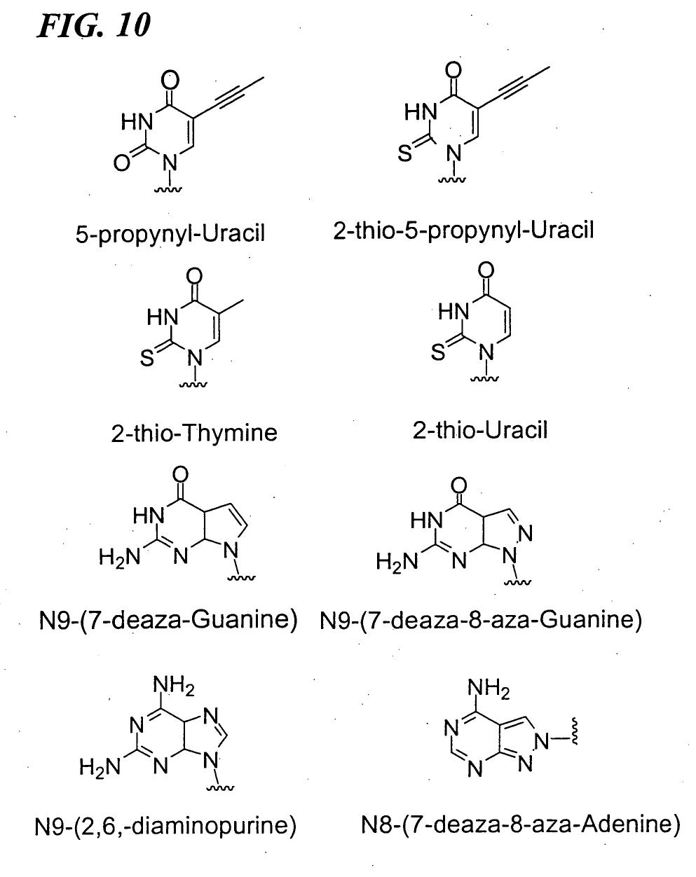

With reference to FIGS. 8A-8C, in each case one of the oligomer blocks comprises a nucleophilic thiol and a leaving group. FIG. 8A illustrates a ligation reaction in accordance with Lu et al., J. Am. Chem. Soc., 118(36):8518-8523 (1996). Reaction of a nucleophilic thiol, such as 2-aminoethly thiol (FIG. 8C), 2-thioacetyl or 3-thiopropionyl, with, for example, either haloacetyl (FIG. 8B), malimido (FIG. 8C) or vinyl will likewise produce a combination oligomer.

Other non-limiting examples of ligation/condensation chemistries suitable for forming the insulating combinatorial oligomers of the invention are widely known in the art, and can include DNA or PNA. For example, a Diels-Alder type reaction (e.g., between maleimide and furan) may be used in this way to form combinatorial oligomers (and also may be used to add dyes or other labels to such oligomers; see, e.g., as discussed, e.g., in Graham et al., Tetrahedron Lett. 43:4785-4788 (2002)).

In some embodiments of the invention, nucleobase protecting groups are used during the oligomer block ligation step. As used herein, a “nucleobase protecting group” is a chemical moiety that is covalently linked to a functional group of a nucleobase to render the functional group unreactive during certain chemical reactions (e.g., during the ligation reaction step). For example, the exocyclic amino groups of adenine, cytosine and guanine are typically protected with a suitable protecting group during de novo chemical oligomer synthesis. The formation of a salt of a functional group to render the group unreactive during a chemical reaction is not a nucleobase-protecting group as used herein, since there is no covalent link.

However, the use of nucleobase protecting groups are not required for use of the present invention, and thus, it is not intended that the use of protecting groups be a limitation of the present invention.

Linker moieties may also help serve as spacers in the insulating combinatorial nucleobase oligomers and oligomer blocks of the invention to minimize the adverse effects of bulky chemical groups or inflexible chemical linkages. One of skill in the art will recognize how to employ linker moieties as spacer moieties in order to preserve or enhance the hybridization or probe/labeling properties of the oligomers of the invention. It is not intended that the invention be limited to any particular spacer structure. However, in one embodiment, when oligomer blocks comprise PNA structures, the primary amine and carbonyl carbon of the N-(2-aminoethyl)-glycine moiety of a PNA subunit are not counted as being atoms of the spacer.

In some embodiments, a spacer or multiple spacer moieties can be positioned between the attachment chemistry and the nucleobases of the oligomer block. In other embodiments, spacers can be used adjacent to a label, for example, to facilitate the chemical coupling of the label to the oligomer, or for the purpose of preserving or enhancing some property associated with the label, e.g., fluorescence. Spacer moieties may also incidentally or intentionally be utilized to improve the water solubility of the insulating combinatorial nucleobase oligomers or oligomer blocks of the invention (see, e.g., Gildea et al., Tett. Lett., 39:7255-7258 [1998]).

Non-limiting examples of spacer/linker moieties suitable for use in this invention consist of: one or more aminoalkyl carboxylic acids (e.g. aminocaproic acid); the side chain of an amino acid (e.g. the side chain of lysine or ornithine); one or more amino acids which occur in natural polypeptides and proteins (e.g. glycine); one or more amino acids not typically found in natural polypeptides and proteins (e.g. ornithine, β-alanine, γ-aminobutyric acid, homocysteine, homoserine, citrulline, canavanine, djenkolic acid, and β-cyanoalanine); one or more O-linker residues; aminooxyalkylacids (e.g. 8-amino-3,6-dioxaoctanoic acid); alkyl diacids (e.g. succinic acid); alkyloxy diacids (e.g. diglycolic acid); alkyldiamines (e.g. 1,8-diamino-3,6-dioxaoctane); the amino acid glycine; the amino acid dimer gly-gly; the amino acid dimer gly-lys; the amino acid dimer lys-gly; the amino acid dimer glu-gly; the amino acid dimer gly-cys; the amino acid dimer cys-gly; and the amino acid dimer asp-gly.

F. Peptide Nucleic Acids

The insulating combinatorial oligomers of the invention can be synthesized from any suitable internucleotide analog. As both enzymatically extendable and enzymatically non-extendable structures find use with the invention, the scaffold that supports the base sequence is not particularly limited, except that the polymeric structure be capable of antiparallel sequence-specific base pairing with a target nucleobase sequence.

A number of non-phosphate, non-ribose containing internucleotide analog structures are known in the art to form suitable nucleobase oligomers, and any such structure finds use with the invention. For discussion of such structures, see, e.g., WO 96/04000.

One non-phosphate, non-ribose structure that has found widespread use in the synthesis of nucleobase polymers is the family of peptide (or polyamide) nucleic acids, commonly referred to as PNA. In these PNA structures, the phospho-diester ribose backbone of DNA or RNA has been replaced with acyclic, achiral, and neutral pseudopeptide polyamide linkages (U.S. Pat. No. 5,539,082; WO 92/20702; Nielsen et al., Science 254:1497-1500 [1991]; Egholm et al., Nature 365:566-568 [1993]). The PNA backbone forms a scaffold for covalently attached nucleobases to form oligomeric structures having defined base sequences. PNA is often characterized as a nucleic acid mimic, rather than a true nucleic acid analog, since its structure is completely synthetic and not derived from nucleic acid.

In some embodiments, a PNA backbone composed of repeating N-(2-aminoethyl)glycine units are used; however, it is not intended that the PNA structures of the invention be limited to this gly PNA linkage structure. The 2-aminoethylglycine polyamide nucleobase polymer has been well-studied and shown to possess exceptional hybridization specificity and affinity. A partial structure of this molecule is shown in FIG. 2 with a carboxyl-terminal amide, and where B is any nucleobase.

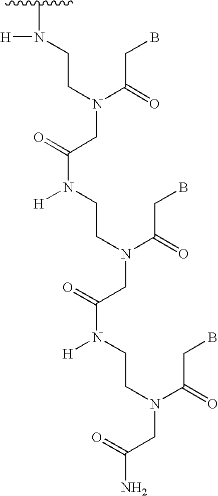

Despite its name, PNA is neither truly a peptide, nor a nucleic acid, nor acidic. PNA is a non-naturally occurring molecule, and is not known to be a substrate for any polymerase enzyme, peptidase or nuclease. Because a PNA is a polyamide, it has a C-terminus (carboxyl terminus) and an N-terminus (amino terminus). For the purposes of the design of a PNA oligomer suitable for antiparallel binding (i.e., hybridization) to a target sequence, the N-terminus of the PNA oligomer nucleobase sequence is equivalent to the 5′-phosphate terminus of a DNA or RNA oligonucleotide, and the C-terminus is equivalent to the 3′-hydroxyl terminus.

As used herein, it is intended that the term “PNA” also include related structures as known in the art, especially other peptide-based nucleic acid mimics (see, e.g., WO 96/04000). Generally, “PNA” means any oligomer or polymer segment (e.g., an oligomer block) comprising two or more PNA subunits (residues), but not nucleic acid subunits (or analogs thereof). The scope of the term “PNA” includes, for example but not limited to, the oligomer or polymer segments described in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053 and 6,107,470; all of which are herein incorporated by reference. The term PNA shall also apply to any oligomer or polymer segment comprising two or more subunits of those nucleic acid mimics described in the following publications: Lagriffoul et al., Bioorganic & Medicinal Chemistry Letters 4:1081-1082 (1994); Petersen et al., Bioorganic & Medicinal Chemistry Letters 6:793-796 (1996); Diderichsen et al., Tett. Lett., 37:475-478 (1996); Fujii et al., Bioorg. Med. Chem. Lett., 7:637-627 (1997); Jordan et al., Bioorg. Med. Chem. Lett., 7:687-690 (1997); Krotz et al., Tett. Lett., 36:6941-6944 (1995); Lagriffoul et al., Bioorg. Med. Chem. Lett., 4:1081-1082 (1994); Diederichsen, Bioorganic & Medicinal Chemistry Letters 7:1743-1746 (1997); Lowe et al., J. Chem. Soc. Perkin Trans., 1, (1997) 1:539-546; Lowe et al., J. Chem. Soc. Perkin Trans., 11:547-554 (1997); Lowe et al., J. Chem. Soc. Perkin Trans. 11:555-560 (1997); Howarth et al., J. Org. Chem., 62:5441-5450 (1997); Altmann et al., Bioorganic & Medicinal Chemistry Letters 7:1119-1122 (1997); Diederichsen, Bioorganic & Med. Chem. Lett., 8:165-168 (1998); Diederichsen et al., Angew. Chem. Int. Ed., 37:302-305 (1998); Cantin et al., Tett. Lett., 38:4211-4214 (1997); Ciapetti et al., Tetrahedron 53:1167-1176 (1997); Lagriffoule et al., Chem. Eur. J, 3:912-919 (1997); Kumar et al., Organic Letters 3(9):1269-1272 (2001); and the Peptide-Based Nucleic Acid Mimics (PENAMs) of Shah et al. as disclosed in WO96/04000. For the avoidance of doubt, the linking of one or more amino acid subunits, or one or more labels or linkers, to a PNA oligomer or segment (e.g., PNA oligomer block) does not produce a PNA chimera.

Methods for the chemical synthesis of PNAs are well known in the art (see, e.g., Nielsen et al., Peptide Nucleic Acids—Protocols and Applications, Horizon Scientific Press, Norfolk England (1999); Nielsen (Ed.), Peptide Nucleic Acids—Methods and Protocols, Humana Press (2002); Hyrup and Nielsen, Bioorg. Med. Chem., 4(1):5-23 (1996); WO 92/20702; WO 92/20703 and U.S. Pat. No. 5,539,082). Chemical assembly of PNA oligomers is analogous to solid phase peptide synthesis, wherein at each cycle of assembly the oligomer possesses a reactive alkyl amino-terminus that is condensed with the next monomer unit to be added to the growing oligomer. Because standard peptide chemistry is utilized, natural and non-natural amino acids can be incorporated into a PNA oligomer using solid phase synthesis, for example. Chemical reagents and instrumentation for support-bound automated chemical synthesis of PNA oligomers are commercially available, and PNA oligomers having custom nucleobase sequences are readily ordered from commercial vendors (e.g., Applied Biosystems, Foster City, Calif.). Labeled PNA oligomers are likewise available from commercial vendors of custom PNA oligomers.

PNA may be synthesized at any scale, from submicromole to millimole, or larger. Most conveniently, PNA is synthesized by solid phase synthesis at the 2 pmole scale, using Fmoc/Bhoc, tBoc/Z, or MMT protecting group monomers on an Expedite Synthesizer (Applied Biosystems) on XAL or PAL support; or on the Model 433A Synthesizer (Applied Biosystems) with MBHA support; or on other automated synthesizers. Because a PNA is a polyamide, it has a carboxy-terminus (i.e., a C-terminus) and an amino-terminus (i.e., an N-terminus). For the purposes of the design of a hybridization probe suitable for antiparallel binding to the target sequence, the N-terminus of the probing nucleobase sequence of the PNA probe is the equivalent of the 5′-hydroxyl terminus of an equivalent DNA or RNA oligonucleotide.

In some embodiments, the PNA oligomers additionally and optionally comprise a linker/spacer moiety, incorporated to improve the solubility of the PNA oligomer, as known in the art (see, WO 99/37670; and Gildea et al., Tetrahedron Letters 39:7255-7258 [1998]). This linker/spacer can be incorporated in an internal, amino-terminal, or carboxy-terminal position, and one or more than one linker/spacer can be incorporated into the oligomer.

In other embodiments, the PNA molecules used in the invention are chiral molecules, i.e., have enantiomeric forms. Peptide nucleic acids having chiral structures are known in the art (D'Costa et al., Tetrahedron Letters 43:883-886 [2002]).

Methods for the labeling and use of PNA oligomers for use as FRET-type probes is well-known in the art, and are described in various sources. See, e.g., PCT WO 99/21881, WO 99/22018 and WO 99/49293.

G. Oligomer Block Libraries

The oligomer blocks used to synthesize the insulating combinatorial nucleobase oligomers of the invention can be of any length. The length of the oligomer blocks is not necessarily limited to a specific number of monomers in the practice of the invention. In one embodiment, for example, the oligomer blocks can be independently selected to comprise between about three and about eight specificity-determining nucleobases. Furthermore, the oligomer blocks can comprise any number of universal nucleobases. For example, in one embodiment, the oligomer blocks can independently comprise between about one and about 10 universal nucleobases.

The oligomer blocks used to synthesize the insulating combinatorial nucleobase oligomers can be presynthesized and maintained in defined libraries. In one embodiment, these oligomer block libraries contain all possible permutations of the four naturally occurring DNA nucleobases (A, C, G and T) at each of the specificity-determining nucleobase positions. In alternative embodiments, not all A-T-G-C permutations are represented in the library. In further embodiments, oligomer block libraries contain all possible permutations of the four naturally occurring RNA nucleobases (A, C, G and uracil) at each of the specificity-determining nucleobase positions. In alternative embodiments, not all A-T-G-uracil permutations are represented in the library.

In other embodiments, the use of non-natural bases within the nucleobase structure is contemplated. The size (i.e., diversity) of an oligomer block library is determined by the number of permutations at a given position raised to the power of the number of variable sites. For example, in the case where the naturally occurring bases are used to form an oligomer block having five specificity-determining nucleobases, a library of oligomer blocks would contain a set of 4⁵ possible variations, or 1,024 possible oligomer block sequences. In addition to the variable nucleobase positions, the blocks will also comprise universal nucleobases. Typically, an oligomer block library will include insulating oligomer nucleobase blocks each having the same number of universal nucleobases. However, oligomer blocks having different numbers of insulating nucleobases may be included in a library of insulating nucleobase oligomer blocks. Thus, for example, an oligomer block library of oligomer blocks each having five specificity-determining nucleobases may have blocks including one, two, three and four universal nucleobases.

In general, an oligomer block library will include oligomer blocks all having the same number of specifity-determining nucleobases. Thus, for example, an oligmoer block library of oligomer blocks having four specificity-determining nucleobases will include 256 (4⁴) different oligomer blocks each having four specificity-determining nucleobases, and will not include oligomer blocks having fewer than, or more than, four specificity-determining nucleobases. However, in embodiments of insulating oligomer block libraries having features of the invention, a library may include insulating nucleobase oligomer blocks having different numbers of specificity-determining nucleobases.

However, it is not intended an oligomer block library of the invention must contain every possible nucleobase sequence permutation for an oligomer block of defined length. That is to say, it is not a requirement that the oligomer block library be a “complete” library. Indeed, there is no requirement that a block library of this invention comprise a complete set of block oligomers or that the sets of block oligomers of a library, or libraries, all be of the same type, length or diversity. Moreover, the oligomer blocks of a library set need not be all of the same length or composition. In one aspect, any collection of more than one oligomer block (i.e., at least two oligomer blocks) can be considered a library. In certain embodiments, a library can comprise one or more non-naturally occurring nucleobases or can comprise only non-naturally occurring nucleobases.

It is also to be understood that the oligomer blocks of a library can be support bound, for example. In one aspect, the library may exist as an array of block oligomers that have been affixed to a solid support. For example, an array of block oligomers may be affixed to a glass plate to form an array suitable for examination in a microarray reader or microarray scanner (see, e.g., DeRisi et al., Science 278:680-686 (1997; Lashkari et al., P.N.A.S. 94:13057-13062 (1997)).

For example, in one aspect of the invention, the oligomer blocks used in the ligation reaction can be chosen from the oligomer blocks that are contained in a block library or libraries to thereby enable the rapid, efficient and/or appropriately scaled synthesis of a insulating combinatorial nucleobase oligomer that is suitable for a chosen application. Thus, the synthesis and use of a library or libraries of oligomer blocks can facilitate the rapid, efficient and/or appropriately scaled synthesis of numerous oligomer blocks of different but defined nucleobase sequence, wherein the number of potential insulating combinatorial nucleobase oligomers of different nucleobase sequence that can be made from a library is determined by the diversity of the library oligomer block set and wherein the diversity of a oligomer block set will depend on the number of oligomers of different nucleobase sequence in the set.

H. Labeling of Insulating Combinatorial Nucleobase Oligomers

Regardless of whether the insulating combinatorial nucleobase oligomers and the oligomer blocks of the invention are synthesized from nucleic acids, modified nucleic acids, nucleic acid analogues (e.g., peptide nucleic acids), or any combination or variation thereof, the molecules that are used to practice of this invention can be labeled with a suitable label/reporter moiety. For example, the insulating combinatorial nucleobase oligomers and the oligomer blocks of the invention may be labeled with a label or with multiple labels selected from the group of labels consisting of dyes, fluorescent labels, luminescent labels, radioactive labels, antigens, haptens, enzymes, enzyme substrates, protecting groups, and chemically reactive groups. Other labels may also be used, in addition to, or in conjunction with, these labels.

As used herein, the term “label” in reference to nucleobase oligomers refers to any moiety that can be attached to the oligomer and: (i) provides a detectable signal, where the signal can be in the visible wavelength spectrum or any other wavelength or particle type, e.g., a radioisotope decay particle; (ii) interacts with a second label to modify the detectable signal provided by the second label, i.e., energy transfer label pairs, e.g., FRET pairs; (iii) stabilizes hybridization, i.e., duplex formation; (iv) confers a capture function, e.g., hydrophobic affinity, antibody/antigen, ionic complexation, or (v) changes a physical property, such as electrophoretic mobility, hydrophobicity, hydrophilicity, solubility, or chromatographic behavior. Labeling can be accomplished using any one of a large number of known techniques employing known labels, linkages, linking groups, reagents, reaction conditions, and analysis and purification methods. Labels include light-emitting or light-absorbing compounds which generate or quench a detectable fluorescent, chemiluminescent, or bioluminescent signal (Kricka, L. in Nonisotopic DNA Probe Techniques (1992), Academic Press, San Diego, pp. 3-28). As used herein, the terms “label” and “reporter” may in some cases be used interchangeably.

It is contemplated that the insulating combinatorial nucleobase oligomers and the oligomer blocks of the invention can be labeled with any labeling moiety or technique currently known in the art for labeling nucleic acids, modified nucleic acids or nucleic acid analogues. It is not intended that the invention be limited in any way to any particular labeling method. Techniques for labeling of nucleic acids, modified nucleic acids and nucleic acid analogues are widely known in the art, and thorough discussion and detailed protocols for labeling are available from many sources. For example, see, “Non-Radioactive Labeling, A Practical Introduction,” Garman, Academic Press, San Diego, Calif. (1997).

A label or reporter moiety can be linked to any position within the insulating combinatorial nucleobase oligomers and the oligomer blocks. A label can reside at a terminus of the oligomer or at a position internal to the oligomer blocks (e.g., within or attached to the nucleobases). The labeling can occur either following synthesis of the complete oligomer or oligomer block, or incorporated during synthesis of the oligomer or oligomer block. Alternatively, a label can be incorporated into or attached to (i.e., integral to) the optional spacer domain located between the linker chemistry and the nucleobases.

Fluorescent reporter dyes useful for labelling biomolecules include fluoresceins (U.S. Pat. Nos. 5,188,934; 6,008,379; 6,020,481), rhodamines (U.S. Pat. Nos. 5,366,860; 5,847,162; 5,936,087; 6,051,719; 6,191,278), benzophenoxazines (U.S. Pat. No. 6,140,500), energy-transfer dye pairs of donors and acceptors (U.S. Pat. Nos. 5,863,727; 5,800,996; 5,945,526), and cyanines (Kubista, WO 97/45539), as well as any other fluorescent label capable of generating a detectable signal. Examples of fluorescein dyes include 6-carboxyfluorescein; 2′,4′,1,4,-tetrachlorofluorescein; and 2′,4′,5′,7′,1,4-hexachlorofluorescein (Menchen, U.S. Pat. No. 5,118,934).

As used herein in reference to a fluorescent label, “quenching” means a decrease in the fluorescence of a fluorescent label (i.e., a fluorescent reporter moiety). A donor moiety may be a fluorophore, and an acceptor moiety (a “quencher” moiety) may be fluorophore or may be a non-fluorescent moiety. In some embodiments, the decrease in fluorescence is caused by fluorescence resonance energy transfer (FRET) associated with a quencher moiety, regardless of the mechanism. Energy transfer may occur between members of a set of energy transfer labels, the set of energy transfer labels having at least one acceptor moiety and at least one donor moiety. In embodiments of the invention, an insulating combinatorial nucleobase oligomer may have at least one energy transfer set of labels. The labels of an energy transfer set may be linked to oligomer termini, or may be linked to sites within an insulating combinatorial nucleobase oligomer. Alternatively, or in addition, an acceptor moiety and a donor moiety may be coupled to different oligomer blocks.

Another class of labels are hybridization-stabilizing moieties which serve to enhance, stabilize, or influence hybridization of duplexes, e.g., intercalators, minor-groove binders, and cross-linking functional groups (Blackburn and Gait, Eds., “DNA and RNA Structure” in Nucleic Acids in Chemistry and Biology, 2^{nd} Edition, (1996) Oxford University Press, pp. 15-81). Yet another class of labels effect the separation or immobilization of a molecule by specific or non-specific capture, for example biotin, digoxigenin, and other haptens (Andrus, “Chemical methods for 5′ non-isotopic labelling of PCR probes and primers” (1995) in PCR 2: A Practical Approach, Oxford University Press, Oxford, pp. 39-54). Suitable haptens include fluorescein, biotin, 2,4-dinitrophenyl, digoxigenin, lipopolysaccharide; apotransferrin; ferrotransferrin; insulin; a cytokine; gp120; β-actin; leukocyte function-associated antigen 1 (LFA-1; CD11a/CD18); Mac-1 (CD11b/CD18); glycophorin; laminin; collagen; fibronectin; vitronectin; an integrin, ankyrin; fibrinogen, Factor X; inter-cellular adhesion molecule 1 (ICAM-1); inter-cellular adhesion molecule 2 (ICAM-2); spectrin, fodrin; CD4; a cytokine receptor; an insulin receptor; a transferrin receptor; Fe⁺⁺⁺; polymyxin B; endotoxin-neutralizing protein (ENP); an antibody-specific antigen; avidin; streptavidin; and biotin. Non-radioactive labelling methods, techniques, and reagents are reviewed in: Non-Radioactive Labelling, A Practical Introduction, Garman (1997) Academic Press, San Diego. In some embodiments, the terms “label” and “reporter” are used interchangeably.

Non-limiting examples of reporter/label moieties suitable for the direct labeling of insulating combinatorial nucleobase oligomers or oligomer blocks include, but are not limited to, a quantum dot, a minor groove binder, a dextran conjugate, a branched nucleic acid detection system, a chromophore, a fluorophore, a quencher, a spin label, a radioisotope, an enzyme, a hapten, an acridinium ester and a chemiluminescent compound. Quenching moieties are also considered labels. Other suitable labeling reagents and preferred methods of label attachment would be recognized by those of ordinary skill in the art. Any examples cited herein are intended to be mererly illustrative and are non-limiting.

Labels

Non-limiting examples of haptens include, but are not limited to, 5(6)-carboxyfluorescein, 2,4-dinitrophenyl, digoxigenin, and biotin.

Non-limiting examples of fluorochromes (fluorophores) include, but are not limited to, 5(6)-carboxyfluorescein (Flu), 2′,4′,1,4,-tetrachlorofluorescein; and 2′,4′,5′,7′,1,4-hexachlorofluorescein, other fluorescein dyes (see, e.g., U.S. Pat. Nos. 5,188,934; 6,008,379; 6,020,481, incorporated herein by reference), 6-((7-amino-4-methylcoumarin-3-acetyl)amino)hexanoic acid (Cou), 5(and 6)-carboxy-X-rhodamine (Rox), other rhodamine dyes (see, e.g., U.S. Pat. Nos. 5,366,860; 5,847,162; 5,936,087; 6,051,719; 6,191,278; 6,248,884, incorporated herein by reference), benzophenoxazines (see, e.g., U.S. Pat. No. 6,140,500, incorporated herein by reference) Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.), other cyanine dyes (Kubista, WO 97/45539), 6-carboxy-4′,5′-dichloro-2′,7′-dimethoxyfluorescein (JOE), 5(6)-carboxy-tetramethyl rhodamine (Tamara), Dye 1 Dye2 or the Alexa dye series (Molecular Probes, Eugene, Oreg.).

Non-limiting examples of enzymes that can be used as labels include, but are not limited to, alkaline phosphatase (AP), horseradish peroxidase (HRP), soy bean peroxidase (SBP), ribonuclease and protease.

The insulating combinatorial nucleobase oligomers of the invention can be used in conjunction with energy transfer label sets to form probes suitable for use in energy tyransfer applications (e.g., FRET probes or probes suitable for use in real-time PCR analysis, i.e., TAQMAN® analysis).

A non-limiting example of aminor groove binder is CDPI3 (see, e.g., WO 01/31063).

Guidance in Label Choices and Protecting Groups

It will be apparent to one of skill in the art that when oligomer blocks are to be ligated in accordance with this invention to thereby produce an insulating combinatorial nucleobase oligomer, the entire nature of the potentially reactive chemical groups of the oligomer blocks should be considered for potential side or cross-reactions. Protecting groups can also be used, as appropriate, to minimize or eliminate potential side or cross-reactions. For example, in the case where oligomer blocks are labeled prior to ligation to form the insulating combinatorial nucleobase oligomer, it is wise to consider the potential for reactivity of functional groups on the label or labels in view of the nature of the various ligation chemistries that can be chosen.

By way of illustration, when performing ligation reactions involving an amino group, carboxylic acid group and water soluble carbodiimide, the labels (e.g., the labels of an energy transfer set) should generally be selected to avoid unprotected reactive amino and carboxylic functional groups to thereby avoid possible side/cross reactions. One of skill in the art will therefore understand how to effect optimal ligation conditions by consideration of the nature of the reactive functional groups of the component parts in view of the nature of the particular ligation chemistry chosen.

In addition to the modification of the oligomer block termini with chemically reactive groups for ligation, the oligomer blocks can be modified and/or protected to thereby incorporate functional groups for labeling or for attachment to surfaces. Such functional groups can be utilized either before or after ligation depending upon factors such as the oligomer synthesis chemistry (e.g., harsh deprotection conditions required that might destroy a label), the ligation chemistry chosen (e.g., functional groups of the desired label might interfere with the condensation chemistry) and the intended use of the functional group (e.g., whether it is intended for labeling or for attachment to a solid support).

PNA Labeling/Modification

In one embodiment, the insulating combinatorial nucleobase oligomer and oligomer blocks comprise PNA. It is contemplated that any reagents or methods that find use with PNA labeling or modification also find use with the present invention. For example, any techniques known in the art for making and using labeled PNA molecules also find use with the insulating combinatorial nucleobase oligomers and oligomer blocks of the invention.

Non-limiting methods for labeling PNAs are well known in the art and are described in a variety of sources. See, e.g., U.S. Pat. Nos. 6,110,676, 6,280,964, WO99/22018, WO99/21881, WO99/37670, WO99/49293 and Nielsen et al., Peptide Nucleic Acids: Protocols and Applications, Horizon Scientific Press, Norfolk, England (1999).

Because the synthetic chemistries of peptides and PNAs are essentially the same, any method commonly used to label a peptide can often be adapted to effect the labeling a PNA oligomer. Generally, the N-terminus of the PNA polymer can be labeled by reaction with a moiety having a carboxylic acid group or activated carboxylic acid group. One or more spacer moieties can optionally be introduced between the labeling moiety and the base-containing subunits of the oligomer (i.e., the nucleobases). Generally, the spacer moiety can be incorporated prior to performing the labeling reaction. If desired, the spacer can be embedded within the label and thereby be incorporated during the labeling reaction.

Typically the C-terminal end of the polymer can be labeled by first attaching a labeled moiety or functional group moiety with the support upon which the PNA oligomer is to be assembled. Next, the first nucleobase-containing synthon of the PNA oligomer can be condensed with the labeled moiety or functional group moiety. Alternatively, one or more spacer moieties (e.g. 8-amino-3,6-dioxaoctanoic acid; the “O-linker”) can be introduced between the label moiety or functional group moiety and the first nucleobase subunit of the oligomer. Once the molecule to be prepared is completely assembled, labeled and/or modified, it can be cleaved from the support, deprotected and purified using standard methodologies.

Alternatively, a functional group on the assembled, or partially assembled, polymer can be introduced while the oligomer is still support-bound. The functional group will then be available for any purpose, including being used to either attach the oligomer to a support or otherwise be reacted with a reporter moiety, including being reacted post-ligation with another oligomer block (by post-ligation we mean at a point after the insulating combinatorial nucleobase oligomer has been fully formed by ligating one or more oligomer blocks). This method, however, requires that an appropriately protected functional group be incorporated into the oligomer during assembly so that after assembly is completed, a reactive functional can be generated. Accordingly, the protected functional group can be attached to any position within the insulating combinatorial nucleobase oligomer or oligomer block, including at the oligomer block termini, at a position internal to the oligomer block, or linked at a position integral to the linker or spacer.

For example, the ε-amino group of a lysine could be protected with a 4-methyl-triphenylmethyl (Mtt), a 4-methoxy-triphenylmethyl (MMT) or a 4,4′-dimethoxytriphenylmethyl (DMT) protecting group. The Mtt, MMT or DMT groups can be removed from the oligomer (assembled using commercially available Fmoc PNA monomers and polystyrene support having a PAL linker; PerSeptive Biosystems, Inc., Framingham, Mass.) by treatment of the synthesis resin under mildly acidic conditions. Consequently, a donor moiety, acceptor moiety or other reporter moiety, for example, can then be condensed with the ε-amino group of the lysine amino acid while the polymer is still support bound. After complete assembly and labeling, the polymer is then cleaved from the support, deprotected and purified using well-known methodologies.

By still another method, the label is attached to the oligomer block after the oligomer block is cleaved from the support, or the label can be attached to the insulating combinatorial nucleobase oligomer after the oligomer blocks are ligated. These methods are preferable where the label is incompatible with the cleavage, deprotection or purification regimes used to manufacture the oligomer. By this method, the insulating combinatorial PNA oligomer or PNA oligomer blocks will generally be labeled in solution by the reaction of a functional group on the polymer and a functional group on the label. Those of ordinary skill in the art will recognize that the composition of the coupling solution will depend on the nature of oligomer and label, such as for example a donor or acceptor moiety. The solution used in the coupling may comprise organic solvent, water or any combination thereof. Generally, the organic solvent will be a polar non-nucleophilic solvent. Non limiting examples of suitable organic solvents include acetonitrile (ACN), tetrahydrofuran, dioxane, methyl sulfoxide, N,N′-dimethylformamide (DMF) and N-methylpyrrolidone (NMP).

The nature of the functional groups on the oligomer and the label is non-limiting. For example, the functional group on the oligomer to be labeled can be a nucleophile (e.g., an amino group) and the functional group on the label can be an electrophile (e.g., a carboxylic acid or activated carboxylic acid). It is contemplated that this location of the nucleophile and electrophile can be inverted such that the functional group on the oligomer can be an electrophile and the functional group on the label can be a nucleophile. Non-limiting examples of activated carboxylic acid functional groups include N-hydroxysuccinimidyl esters. In aqueous solutions, the carboxylic acid group of either of the PNA or label (depending on the nature of the components chosen) can be activated with a water soluble carbodiimide, for example. The reagent, 1-ethyl-3-(3-dimethylamino-propyl)carbodiimide hydrochloride (EDC), is a commercially available reagent sold specifically for aqueous amide forming condensation reactions.

I. Chimeric Insulating Combinatorial Nucleobase Oligomers and Oligomer Blocks

It is contemplated that the oligomers of the invention, including the insulating combinatorial nucleobase oligomers as well as the separate oligomer blocks, can be chimeric in nature. That is to say, the insulating combinatorial nucleobase oligomers and/or the individual oligomer blocks can comprise nucleobases or differing structures.

For example, in the case of insulating combinatorial nucleobase oligomers, the oligomers, can comprise two or more oligomer blocks, where the nucleobase structure of the different oligomer blocks are of a different structure. For example, an insulating combinatorial nucleobase oligomer can be synthesized from two oligomer blocks, where one oligomer block is sythesized from PNA and the second oligomer block can be synthesized from deoxyribonucleotides (DNA). Furthermore, it is contemplated that a single oligomer block can by itself be chimeric. For example, one oligomer block can comprise PNA nucleobases as well as DNA nucleobases.

As a non-limiting example, chimeric nucleobase structures comprising PNA, e.g., chimeras comprising both PNA and polynucleotide structures, are known in the art. The synthesis, labeling and modification of these chimeras can utilize methods known to those of skill in the art. See, e.g., WO96/40709, now issued U.S. Pat. No. 6,063,569, herein incorporated by reference. Moreover, the methods described above for PNA synthesis and labeling often can be used for modifying the PNA portion of a PNA chimera, for example. Additionally, well-known methods for the synthesis and labeling of nucleic acids can often be used for modifying the nucleic acid portion of a PNA chimera. Exemplary methods can be found in U.S. Pat. Nos. 5,476,925, 5,453,496, 5,446,137, 5,419,966, 5,391,723, 5,391,667, 5,380,833, 5,348,868, 5,281,701, 5,278,302, 5,262,530, 5,243,038, 5,218,103, 5,204,456, 5,204,455, 5,198, 540, 5,175,209, 5,164,491, 5,112,962, 5,071,974, 5,047,524, 4,980,460, 4,923,901, 4,786,724, 4,725,677, 4,659,774, 4,500,707, 4,458,066, and 4,415,732; each of which are herein incorporated by reference.

J. Articles of Manufacture

The present invention provides articles of manufacture (e.g., kits) comprising at least one insulating combinatorial oligomer or oligomer block of the invention. In certain embodiments, kits serve to facilitate the performance of a process, method, assay, analysis or manipulation of interest by assembling two or more components used to carry out the methods. Kits can contain any chemical reagent, enzyme, or equipment required for use of the method. In certain embodiments, kits contain components in pre-measured amounts to minimize the need for measurements by end-users. In certain embodiments, kits include instructions for performing one or more methods of the invention. In certain embodiments, the kit components are optimized to operate in conjunction with one another.

When used in kits of the invention, the insulating combinatorial oligomer can be made sequence-specific for any given target sequence, and can be labeled or unlabeled. If the insulating combinatorial oligomer is labeled, the label chosen will be suitable for use in the intended application. The insulating combinatorial oligomer can be prepared from any suitable polynucleobase, e.g., from PNA. The oligomers of the invention can be packaged in suitable containers, such as tubes or ampules, and can be packaged in a dried (e.g., lyophilized) form, or in an aqueous form. If necessary, the articles of manufacture in the kits can be chilled or frozen during shipping and/or storage. Any article of manufacture comprising the insulating combinatorial oligomer of the invention can further include a description of the product, specifications of the product, or instructions for use of the product.

In addition, kits of the present invention can also include, for example but not limited to, apparatus and reagents for sample collection and/or purification, apparatus and reagents for product collection and/or purification, sample tubes, holders, trays, racks, dishes, plates, instructions to the kit user, solutions, buffers or other chemical reagents, suitable samples to be used for standardization, normalization, and/or control samples. Kits of the present invention can also be packaged for convenient storage and shipping, for example, in a box having a lid.

The insulating combinatorial oligomers provided in the kits may or may not be labeled. In other embodiments, the invention provides kits comprising unlabeled insulating combinatorial oligomers as well as means for labeling the oligomers. In other embodiments, the invention provides kits comprising labeled or unlabeled insulating combinatorial oligomers as well as means (e.g., apparatus and/or reagents) for the visualization or detection the oligomers.

The invention also provides kits to facilitate use of the insulating combinatorial oligomers of the invention in various methods, e.g., any method that involves sequence-specific hybridization. Materials and reagents to carry out these methods can be provided in kits to facilitate execution of the methods. A kit of the invention comprises at least one insulating combinatorial oligomer, and optionally can additionally comprise and number of additional components, including but not limited to (i) one or more buffers; (ii) one or more nucleotide triphosphates; (iii) a nucleic acid amplification master mix; (iv) one or more polymerase enzymes, or (v) reagents or equipment suitable for the isolation/purification of a nucleic acid product. In one embodiment, the kit comprises at least two oligonucleotide primers suitable for use as primers in a PCR reaction.

In some embodiments, the present invention provides kits for conducting TAQMAN® real-time PCR analysis. These kits can include, for example but not limited to, reagents for the collection of a sample, a reverse transcriptase, primer suitable for reverse transcriptase initiation and first strand cDNA synthesis, at least one suitable blocking nucleobase oligomer, primer suitable for second strand cDNA synthesis, a DNA-dependent DNA polymerase, free deoxyribonucleotide triphosphates, and reagents suitable for the isolation/purification of the cDNA molecules produced by the reaction.

In one embodiment providing kits of the invention, a single nucleobase oligomer is provided that is specific for a single target sequence. In other embodiments, multiple nucleobase oligomers specific for a plurality of targets are provided in the kit. For example, a set of two insulating combinatorial oligomers can be provided in a single kit, where the two oligomers can be used as primers in a nucleic acid amplification amplification reaction, such as a PCR reaction. In some embodiments, kits are provided having the insulating combinatorial oligomers of the invention affixed to a solid phase or surface. In certain embodiments, the kits of the invention may be used to sequence at least one target nucleic acid template.

In still other embodiments, the present invention provides kits for the analysis of gene expression using the insulating combinatorial oligomers of the invention. These kits can include multiple insulating combinatorial oligomers of the invention affixed to a suitable array or chip configuration, as well as reagents required for the detection/visualization of hybridized complexes.

K. Labeling of Insulating Combinatorial Nucleobase Oligomers for use in Energy Transfer Application (e.g., FRET or TAQMAN®)

Pairs of labels that constitute energy transfer label sets (or energy transfer label pairs) also find use with the insulating combinatorial nucleobase oligomers of the invention in energy transfer applications (e.g., fluorescence resonance energy transfer or FRET probes or probes suitable for use in real-time PCR analysis, i.e., TAQMAN® analysis). Energy transfer probe sets have found widespread and diverse uses in cellular/molecular biological studies, and protocols for their synthesis and use are widely known in the art. See, for example, WO 99/21881, WO 99/22018 and WO 99/49293.

Generally, an energy transfer pair refers to at least two labels where the emission of one label (sometimes called the “donor” or “quencher”) affects the intensity of a second label (sometimes called the “acceptor”). In one embodiment, both the donor moiety(ies) and acceptor moiety(ies) are fluorophores, and the labels comprise a FRET pair. The labels of the energy transfer set can be linked to the insulating combinatorial nucleobase oligomers at the oligomer block termini or linked at sites within the oligomer blocks or elsewhere in the insulating combinatorial nucleobase oligomers (e.g., integral to the spacer moiety). In one embodiment, each of two labels of an energy transfer set can be linked at the distal-most termini of the combination oligomer. In one embodiment, one oligomer block comprises the donor moiety and a second oligomer block comprises the acceptor moiety.

In this application, the energy transfer set comprising at least one energy transfer donor and at least one energy transfer acceptor moiety. Often, the energy transfer set will include a single donor moiety and a single acceptor moiety, but this is not a limitation. An energy transfer set may contain more than one donor moiety and/or more than one acceptor moiety. The donor and acceptor moieties operate such that one or more acceptor moieties accepts energy transferred from the one or more donor moieties or otherwise quenches the signal from the donor moiety or moieties. In one embodiment, both the donor moiety(ies) and acceptor moiety(ies) are fluorophores, and the labels comprise a FRET pair. Although a variety of fluorophores with suitable spectral properties might operate as energy transfer acceptors, the acceptor moiety can also be a non-fluorescent quencher-type moiety.

Non-limiting examples of quenching moieties include but are not limited to diazo-containing moieties such as aryldiazo compounds, e.g., 4-((-4-(dimethylamino)phenyl)azo) benzoic acid (dabcyl), dabsyl, homologs containing one more additional diazo and/or aryl groups; e.g., Fast Black, (see, e.g., U.S. Pat. No. 6,117,986), cyanine dyes (see, e.g., U.S. Pat. No. 6,080,868) and other chromophores such as anthraquinone, malachite green, nitrothiazole, and nitroimidazole compounds.

Transfer of energy between donor and acceptor moieties may occur through any energy transfer process, such as through the collision of the closely associated moieties of an energy transfer set(s) or through a non-radiative process such as FRET. For FRET to occur, transfer of energy between donor and acceptor moieties requires that the moieties be close in proximity and that the emission spectrum of a donor(s) have substantial overlap with the absorption spectrum of the acceptor(s) (see Yaron et al., Analytical Biochemistry 95:228-235 (1979) and particularly page 232, col. 1 through page 234, col. 1). Alternatively, collision mediated (radiationless) energy transfer may occur between very closely associated donor and acceptor moieties whether or not the emission spectrum of a donor moiety(ies) has a substantial overlap with the absorption spectrum of the acceptor moiety(ies) (see Yaron et al., Analytical Biochemistry 95:228-235 (1979) and particularly page 229, col. 1 through page 232, col. 1). This process is referred to as intramolecular collision since it is believed that quenching is caused by the direct contact of the donor and acceptor moieties (see Yaron et al., supra). It is to be understood that any reference to energy transfer in the instant application encompasses all of these mechanistically-distinct phenomena. It is also to be understood that energy transfer can occur though more than one energy transfer process simultaneously and that the change in detectable signal can be a measure of the activity of two or more energy transfer processes. Accordingly, the mechanism of energy transfer is not a limitation of this invention. Indeed, an understanding of the mechanism or mechanisms by which energy transfer works is not required to make or use the invention.

Energy transfer pairs can be used to detect/monitor nucleobase hybridization between an insulating combinatorial nucleobase oligomer of the invention and a target. When used in this manner, the insulating combinatorial nucleobase oligomers can be labeled with a suitable energy transfer pair prior to use as a probe. Suitable energy transfer pairs to use in this type of application are known in the art, where such a probe is sometimes termed a “linear beacon” or a “molecular beacon” (see, e.g., WO99/21881).

The formation of a hybridization complex between a suitably labeled insulating combinatorial nucleobase oligomer and a target sequence can be monitored by measuring at least one physical property of at least one member of the energy transfer set that is detectably different when the hybridization complex is formed as compared with when the insulating combinatorial nucleobase oligomer exists in a non-hybridized state. This change in detectable signal results from the change in efficiency of energy transfer between donor and acceptor moieties caused by hybridization of the combination oligomer to the target sequence.

For example, the means of detection can involve measuring fluorescence of a donor or acceptor fluorophore of an energy transfer set. In one embodiment, the energy transfer set comprises at least one donor fluorophore and at least one acceptor (fluorescent or non-fluorescent) quencher such that the measure of fluorescence of the donor fluorophore can be used to detect, identify or quantitate hybridization of the insulating combinatorial nucleobase oligomer to the target sequence. For example, there may be a measurable increase in fluorescence of the donor fluorophore upon the hybridization of the combination oligomer to a target sequence.

In another embodiment, the labels of the energy transfer pair reside on different insulating combinatorial nucleobase oligomers of the present invention, where one oligomer is labeled solely with a quencher moiety, and one oligomer is labeled solely with an acceptor moiety, and further where the oligomers have a domain of overlapping nucleobase complementarity and one oligomer is further specific for a target that is not the remaining oligomer. This type of label system has various uses, and is known in the art (see, e.g., WO99/49293). This labeling technique can be used in conjunction with the novel insulating combinatorial nucleobase oligomers of the present invention.

In this system, when a complex comprising the oligomer, quencher and target is formed, at least one donor moiety on the target is brought within sufficient proximity to at least one acceptor moiety on a second insulating combinatorial nucleobase oligomer bound to a second target. Since the donor and acceptor moieties of the set are in close proximity, transfer of energy occurs between moieties of the energy transfer set. However, when one of the detection complexes dissociates, as for example when a polymerase copies one of the strands of the detection complex, the donor and acceptor moieties no longer interact sufficiently to cause substantial transfer of energy from the donor and acceptor moieties of the energy transfer set and there is a correlating change in detectable signal from the donor and/or acceptor moieties of the energy transfer set. Consequently, the formation or dissociation of a complex comprising the insulating combinatorial nucleobase oligomer can be determined by measuring at least one physical property of at least one member of the energy transfer set that is detectably different when the complex is formed as compared with when the component the labeled insulating combinatorial nucleobase oligomers exist independently and are unassociated.

L. Applications and Methods of Use

The compositions and methods of the present invention finds use in a variety of applications. Indeed, the insulating combinatorial oligomers of the present invention find use in any application where any other nucleobase oligomer is used in a hybridization protocol (i.e., as a probe or a primer). For example, the compositions and methods of the invention find use in the analysis of gene expression. It is not intended that the invention find use in only the few applications discussed herein, as one familiar with the art will immediately recognize a variety of uses for insulating combinatorial oligomers of the invention. The uses cited herein are intended to be exemplary and not limiting, and such examples are not exhaustive. It is understood that use of the invention is not limited to any particular application cited herein, as the invention finds use with any protocol that incorporates oligomeric nucleobase sequences as probes or primers.

When used as probes or primers, it is a requirement that the insulating combinatorial nucleobase oligomers hybridize to a target sequence with sequence specificity. Thus, when used as a probe, there are no additional limitations on specific features of the insulating combinatorial nucleobase oligomer. However, when used as a primer, it is a requirement that the insulating combinatorial nucleobase oligomer be extendable by at least one polymerase enzyme.

The insulating combinatorial oligomers of the invention find particular use in high throughput applications that utilize large numbers (hundreds or even thousands or millions) of oligomer sequences having defined specificities.

Insulating Combinatorial Nucleobase Oligomers as Indicators of Hybridization

In one embodiment, the invention provides compositions and methods for detecting the presence of a target nucleobase sequence in a sample using a suitably labeled insulating combinatorial nucleobase oligomer of the invention.

The insulating combinatorial nucleobase oligomer may comprise an energy transfer set of labels (e.g., a FRET-pair of labels) as known in the art, such that at least one acceptor moiety of the energy transfer set is linked to one of the oligomer blocks while at least one donor moiety is linked to another oligomer block, wherein labels are linked to the insulating combinatorial nucleobase oligomer at positions that facilitate a change in detectable signal of at least one of the labels when the insulating combinatorial nucleobase oligomer is sequence specifically hybridized to a target. Methods for the synthesis and use of FRET-type probes to indicate hybridization to a target sequence are known in the art. See, e.g., WO 99/21881, WO 99/22018 and WO 99/49293.

Insulating Combinatorial Nucleobase Oligomers Comprising Enzyme Cleavage Sites

In one embodiment, an insulating combinatorial nucleobase oligomer of the invention can be engineered to contain an enzyme cleavage site in the attachment or the linker(s), where the cleavage site is protected from cleavage upon the hybridization of the oligomer with a target sequence. Thus, this invention provides a method for determining whether or not an insulating combinatorial oligomer is bound to a target sequence.

In this method, the insulating combinatorial nucleobase oligomer comprising the enzyme cleavage site is labeled with a suitable FRET pair of labels. The labeled oligomer and the possible target are combined under suitable binding conditions to thereby permit hybridization to form a complex.

Following the hybridization reaction, the reaction mix is treated with an enzyme suitable for cleaving the cleavage site under suitable enzyme cleaving conditions. Suitable enzyme cleavage conditions are those conditions under which the enzyme operates to act on a substrate. Numerous enzymes are commercially available for such use, and product literature from the commercial vendor will provide information on suitable enzyme cleavage conditions.

If the oligomer is hybridized to its target sequence, the cleavage site is protected from the enzyme, and the combinatorial oligomer remains uncleaved. However, if the probe is unhybridized to a target, the enzyme cleaves the oligomer, resulting in a change in FRET label intensity compared to the hybridized state.

According to the method, the enzyme will not substantially cleave the insulating combinatorial nucleobase oligomer provided that it binds to a target sequence. Thus, binding protects the oligomer from substantial degradation by the enzyme. Consequently, if the assay determines that the oligomer is not substantially degraded, it must have bound to the target sequence. Conversely, where the oligomer was not protected from degradation, it can be concluded that the potential target sequence was not present. Also, it is understood that since such an assay relies upon an enzymatic event, quantitation of the target sequence can be determined by quantitating the enzyme activity.

When the method involves the binding of a insulating combinatorial nucleobase oligomer to a target sequence, the hybridization will occur under suitable hybridization conditions wherein the target sequence can be in higher concentration than the labeled oligomer so that essentially all of the available oligomer is sequence specifically bound if the target sequence of contiguous nucleobases is present.

Insulating combinatorial nucleobase oligomers of the invention can be designed to comprise an enzyme cleavage site in the linker sequence(s) within the oligomer block(s) or as part of the attachment moieties, for example. Thus, by incorporating such an engineered linker, the present invention provides methods, for example, for determining whether or not a potential target binding partner is present in a sample, and in what amount.

Non-limiting examples of the linker or spacer that comprises a cleavage site include, but are not limited to: lys-X, arg-X, Glu-X, asp-X, asn-X, phe-X, leu-X, lys-gly, arg-gly, glu-gly and asp-glu, wherein X is any naturally occurring amino acid. A list of non-limiting examples of enzymes suitable for cleaving one or more of these substrates include: endoprotinase Glu-C (EC 3.4.21.19), Lys-C (EC 3.4.21.50), Arg-C (EC 3.4.22.8), Asp-N (EC 3.4.24.33), papain (EC 3.4.22.2), pepsin (EC 3.4.23.1), proteinase K (3.4.21.14), chymotrypsin (EC 3.4.21.1) and trypsin (3.4.21.4).

Real-Time Monitoring of PCR Products

The general application of energy transfer (e.g., FRET) labels in conjunction with the insulating combinatorial nucleobase oligomers and oligomer blocks of the invention are discussed above. Another application of energy transfer labeling is the synthesis of probes suitable for real-time monitoring of the accumulation of PCR products, i.e., TAQMAN® analysis.

The insulating combinatorial oligomers of the invention find use FRET-type probes in real-time quantitative PCR analysis. Real-time PCR analysis refers to the monitoring of accumulating PCR products (also known as a fluorogenic 5′ nuclease assay, i.e., TAQMAN® analysis. Methods for the synthesis and use of TAQMAN® probes are well known in the art. See, for example, Holland et al., Proc. Natl. Acad. Sci. USA 88:7276-7280 [1991] and Heid et al., Genome Research 6:986-994 [1996]).

In general, the TAQMAN® PCR procedure uses two oligonucleotide primers to generate an amplicon from a template typical of a PCR reaction. A third non-priming nucleobase oligomer (not necessarily a nucleotide oligomer) is also included in the reaction (the TAQMAN® probe). This probe has a structure that is non-extendible by Taq DNA polymerase enzyme, and is designed to hybridize to nucleotide sequence located between the two PCR primers. The TAQMAN® probe is labeled with a reporter fluorescent dye and a quencher fluorescent dye on opposite termini. The laser-induced emission from the reporter dye is quenched by the quenching dye when the two dyes are located close together, as they are when the probe is annealed to the PCR amplicon.

The TAQMAN® PCR reaction uses a thermostable DNA-dependent DNA polymerase (e.g., Taq DNA polymerase) that retains 5′-3′ nuclease activity despite exposure to elevated temperatures. During the PCR amplification reaction, the Taq DNA polymerase cleaves the labeled probe that is hybridized to the amplicon. The resultant probe fragments disassociate in solution, and signal from the released reporter dye is free from the quenching effect of the second fluorophore. One molecule of reporter dye is liberated for each new molecule synthesized, and detection of the unquenched reporteP dye provides the basis for quantitative interpretation of the data, such that the amount of released fluorescent reporter dye is directly proportional to the amount of amplicon product.

TAQMAN® assay data are expressed as the threshold cycle (CT), which is the minimal number of PCR cycles required to achieve a statistically significant detectable level of fluorescence from the reporter dye. As discussed above, fluorescence values are recorded during every PCR cycle and represent the amount of product amplified to that point in the amplification reaction.

TAQMAN® RT-PCR can be performed using commercially available equipment, such as, for example, ABI PRISM® 7700 Sequence Detection System (Applied Biosystems, Foster City, Calif.), or Lightcycler (Roche Molecular Biochemicals, Mannheim, Germany). The system consists of a thermocycler, laser, charge-coupled device (CCD), camera and computer. The ABI PRISM® 7700 Sequence Detection System amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

Analysis of Gene Expression

The insulating combinatorial nucleobase oligomers provided by the present invention find use in hybridization assays, e.g., in the analysis of gene expression. The insulating combinatorial oligomers of the invention are used as probes in two general capacities. First the insulating combinatorial oligomer of the invention can be labeled and used to detect a target polynucleotide. Second, the insulating combinatorial oligomer of the invention can be immobilized to a solid phase and used in an array or chip type gene expression analysis system. It is not intended that the insulating combinatorial oligomers of the present invention be limited to use in any particular hybridization format, protocol or conditions, as one familiar with the art is familiar with a variety of hybridization protocols, and recognizes well the advantages of the present invention as they apply to many hybridization formats.

In the first aspect, the insulating combinatorial oligomer of the invention is labeled prior to hybridization and use as a probe. It is not intended that the present invention place any restriction on how the labeled probe is used. As used herein, the term “label” refers to any moiety that allows isolation, cloning, detection, visualization, or quantitation of a target nucleotide sequence. The label that is covalently attached to an oligomer may be detectable by itself (e.g., fluorescein or a radioisotope), or conversely, may not be directly visualized until interaction with a secondary reagent (e.g., a biotin/strepavidin coupled dye, or a conjugated enzyme that requires the presence of a chromogenic substrate). The labeled insulating combinatorial oligomer when in a complex (e.g., a duplex) with a target sequence can be detected using a suitable method, for example but not limited to radiometric detection, colorimetric determinations, fluorescence, chemiluminescence, bioluminescence and enzyme-coupled assays. Numerous oligomer labeling/detection techniques are widely known in the art, all of which find use with the present invention. It is not intended that the present invention be limited to any particular labeling method.

In the second aspect, the hybridization reactions take place in high throughput formats, as known in the art. Generally, the high throughput hybridization formats use a probe (i.e., a insulating combinatorial oligomer of the invention) that is affixed to a solid support. The solid support can be any composition and configuration, and includes organic and inorganic supports, and can comprise beads, spheres, particles, granules, planar or non-planar surfaces, and/or in the form of wells, dishes, plates, slides, wafers or any other kind of support. In some embodiments, the structure and configuration of the solid support is designed to facilitate robotic automation technology. The steps of detecting, measuring and/or quantitating can also be done using automation technology.

In some embodiments, the hybridization format is an “array”, “microarray”, “chip” or “biochip” as widely known in the art (see, e.g., Ausubel et al. (eds.), Current Protocols in Molecular Biology, Chapter 22, “Nucleic Acid Arrays,” John Wiley & Sons, Inc., New York [1994]; and M. Schena, (ed.), Microarray Biochip Technology, BioTechnique Books, Eaton Publishing, Natick, Mass. [2000]). In general, array formats facilitate automated analysis of large numbers of samples and/or have a large number of addressable locations, so that patterns of gene expression for a very large number of genes can be studied very rapidly. The insulating combinatorial oligomers of the invention, when used as probes, find use with array formats, and it is not intended that the insulating combinatorial oligomer probes of the present invention be limited to use in any particular array or hybridization format.

The use of polynucleotide samples in hybridization assays typically necessitate the labeling of the polynucleotide pool prior to hybridization, so that an interaction between the immobilized probe and the target can be detected. A variety of polynucleotide labeling techniques are known in the art, and it is not intended that the present invention be limited to any particular polynucleotide labeling method. The labeled polynucleotide sample permits the detection of those species that are in a duplex with a probe affixed to a solid support, such as in a microarray. A labeled polynucleotide in a duplex with the affixed probe can be detected using a suitable detection method.

In one embodiment of the invention, the labeling of the polynucleotide pool (comprising either RNA or DNA molecules) is accomplished by incorporating a suitable label into the nascent polynucleotide molecules at the time of synthesis. For example, dye-coupled UTP can be incorporated into a nascent RNA chain. In an alternative embodiment, the labeling of the polynucleotide pool is accomplished after the polynucleotide pool is synthesized. In these embodiments, the RNA or DNA molecules are labeled using a suitable label that is coupled (i.e., conjugated or otherwise covalently attached) to the polynucleotides after chain synthesis.

In still other embodiments, an unlabeled pool of polynucleotides in a sample can be used directly in hybridization or gene expression analysis using methods that do not required a labeling step. For example, duplex formation with an affixed probe can be detected using surface plasmon resonance (SPR). See, e.g., SPREETATM SPR biosensor (Texas Instruments, Dallas, Tex.), and BIACORE® 2000 (BIACORE®, Uppsala, Sweden). Resonant light scattering methods can also be used to detect duplex formation in a hybridization analysis using probes that have not been otherwise labeled (Lu et al., Sensors 1:148-160 [2001]).

It is not intended that the present invention be limited to any particular labeling, probing, or hybridization method. One skilled in the art is familiar with a wide variety of such protocols and reagents, all of which find use with the insulating combinatorial oligomers of present invention.

Use in Hybridization Reactions

The insulating combinatorial nucleobase oligomers and oligomer blocks of the invention find use in any method involving hybridization, i.e., the forming of a complex between two complementary nucleobase sequences. The complementarity need not be 100%, as effective hybridizations can occur when there is less than 100% complementarity.

The potential uses of the insulating combinatorial nucleobase oligomers and oligomer blocks of the invention are not in any way limited. Thus, one familiar with the art recognizes that the specific conditions to be used in hybridization reactions as practiced using compositions of the invention are similarly unlimited, and are dependent on the particular application and the primary sequence of the oligomers used. A wide variety of sources are available that describe hybridization conditions for particular application; see, e.g., Ausubel et al. (eds.), Current Protocols in Molecular Biology, Chapter 22, “Nucleic Acid Arrays,” John Wiley & Sons, Inc., New York [1994]; and M. Schena, (ed.), Microarray Biochip Technology, BioTechnique Books, Eaton Publishing, Natick, Mass. [2000].

One of ordinary skill in the art appreciates that the stringency of a particular hybridization reaction is dependent on many variables. Although the art refers to “low stringency” or “high stringency,” defining strict conditions of low or high stringency that universally apply to any and all hybridization reactions is impractical if not impossible.

A more useful definition of “stringency” for use in a particular hybridization reaction is to define a given set of hybridization conditions as more or less stringent than a second set of hybridization conditions in the same experimental system. One familiar with the art will know that a variety of factors determine stringency, including but not limited to salt concentration (i.e., ionic strength), hybridization temperature, detergent concentration, pH, the presence/concentration of chemical denaturants (e.g., formamide), and the presence/concentration of chaotropic agents (e.g., urea). Optimal stringency for a particular oligomer is often found by the well-known technique of fixing several of the aforementioned stringency factors and then determining the effect of varying a single stringency factor. Generally, these same stringency factors apply in controlling hybridization stringency for any nucleobase structure. One exception is the use of PNA oligomeric structures in hybridization reactions with nucleic acids, as PNA hybridization stability is fairly independent of ionic strength. Optimal or suitable stringency for an assay may be experimentally determined by examination of each stringency factor until the desired degree of discrimination is achieved.

Immobilization on a Solid Support (e.g., Arrays)

In one aspect, the invention pertains to compositions and methods for making and using insulating combinatorial nucleobase oligomers that are affixed to a solid support. A wide variety of solid supports find use with the invention, and it is not intended that the invention be limited to the use of any particular type of solid support. Similarly, it is not intended that the manner in which the insulating combinatorial nucleobase oligomers are affixed to the solid support be limited in any way.

In one embodiment, the support-bound insulating combinatorial nucleobase oligomers form an array (e.g., a chip) of oligomers. Detailed methods for making and using arrays comprising polymeric nucleobase structures (e.g., nucleic acid, modified nucleic acids, nucleic acid analogs, or chimeric structures) are well-known in the art and are described in many sources. See, e.g., Ausubel et al. (eds.), Current Protocols in Molecular Biology, Chapter 22, “Nucleic Acid Arrays,” John Wiley & Sons, Inc., New York [1994]; and M. Schena, (ed.), Microarray Biochip Technology, BioTechnique Books, Eaton Publishing, Natick, Mass. [2000]. Any methods for the synthesis and use of nucleic acids, modified nucleic acids and nucleic acid analogues with solid supports, and more specifically arrays, are applicable for use with the present invention.

There exist various methods by which these arrays can be synthesized. In one aspect, a presynthesized insulating combinatorial nucleobase oligomer of the present invention can be affixed to the solid support of the array using any method known in the art (e.g., UV crosslinking).

Alternatively, the insulating combinatorial nucleobase oligomer can be synthesized by ligating two or more oligomer blocks directly on the solid support. For example, one oligomer block can be attached to a solid support in such a way that the linker chemistry is available for subsequent chemical reaction. Once affixed to the support, a preformed second oligomer block can be reacted directly on the array resulting in covalent ligation with the first oligomer block to form the complete insulating combinatorial nucleobase oligomer that is attached to the array. This method can further comprises repeating the ligation step with one or more additional oligomer blocks at one or more different sites in the array until the desired array of insulating combinatorial nucleobase oligomers is constructed.

In another aspect, this invention pertains to forming an array where a functional group on a preformed insulating combinatorial nucleobase oligomer is reacted with and forms a bond with a second functional group that is attached to the solid support, thereby covalently attaching the insulating combinatorial nucleobase oligomer to the surface of the solid support. The method further comprises repeating the oligomer attachment step with one or more different insulating combinatorial nucleobase oligomers at one or more different sites until the desired array of oligomers is achieved.

Because the location and sequence of each support bound oligomer is known, arrays can be used to simultaneously detect, identify and/or quantitate the presence or amount of one or more target sequences in a sample. For example, a target sequence can be captured by the complementary insulating combinatorial nucleobase oligomer on the array surface and then the complex containing the target sequence can be detected. Since the sequence of the insulating combinatorial nucleobase oligomer is known at each location on the surface of the array, the sequence of target sequence(s) can be directly detected, identified and/or quantitated by determining the location of a detectable signal generated on the array. Thus, arrays are useful in diagnostic applications or in screening compounds, e.g., during development of therapeutic compounds.

In one embodiment, the insulating combinatorial nucleobase oligomers and/or oligomer blocks can be immobilized to a surface using the well known process of UV-crosslinking.

In another embodiment, the oligomer blocks can be synthesized on the surface in a manner suitable for deprotection but not cleavage from the synthesis support (see, e.g., Weiler et al., Hybridization based DNA screening on peptide nucleic acid (PNA) oligomer arrays,” Nucl. Acids Res., 25(14):2792-2799 (1997)). In still another embodiment, one or more insulating combinatorial nucleobase oligomers or oligomer blocks can be covalently linked to a surface by the reaction of a suitable functional group on the oligomer with a functional group of the surface (see, e.g., Geiger et al., PNA Array technology in molecular diagnostics, Nucleosides & Nucleotides 17(9-11):1717-1724 (1998)). This method is advantageous since the oligomers immobilized on the surface can be highly purified and attached using a defined chemistry, thereby possibly minimizing or eliminating non-specific interactions.

Methods for the chemical attachment of insulating combinatorial nucleobase oligomer and/or oligomer blocks to solid support surfaces can involve the reaction of a nucleophilic group, (e.g., an amine or thiol) of the oligomer to be immobilized, with an electrophilic group on the solid support surface. Alternatively, the nucleophile can be present on the support and the electrophile (e.g., activated carboxylic acid) can be present on the oligomer. In one embodiment, in the case where the oigomer blocks comprise PNA, the PNA used may or may not require modification prior to the immobilization reaction because PNA possesses an amino terminus in its structure.

Conditions suitable for the immobilization of a insulating combinatorial nucleobase oligomer or oligomer block to a surface are widely known in the art, and will generally be similar to those conditions suitable for the labeling of an oligomer. The immobilization reaction to a solid support is anaologous to a labeling reaction, where the label is substituted with the surface to which the polymer is to be linked. It is not intended that the invention be limited to any particular immobilization chemistry or method.

Numerous types of solid supports derivatized with amino groups, carboxylic acid groups, isocyantes, isothiocyanates and malimide groups are commercially available. Non-limiting examples of suitable solid supports include chips of any type (e.g., arrays), membranes, glass, controlled pore glass, polystyrene particles (beads), silica and gold nanoparticles. All of the above recited methods of immobilization are not intended to be limiting in any way but are merely provided by way of illustration.

Detection/Identification of Biological Organisms

The insulating combinatorial nucleobase oligomers of the invention find use in the detection, identification and/or enumeration of biological organisms, and especially, pathogens. Such organisms can include viruses, bacteria and eucarya in food, beverages, water, pharmaceutical products, personal care products, dairy products or in samples of plant, animal, human or environmental origin. The insulating combinatorial nucleobase oligomers find use in the analysis of raw materials, equipment, products or processes used to manufacture or store food, beverages, water, pharmaceutical products, personal care products dairy products or environmental samples. Additionally, the insulating combinatorial nucleobase oligomers find use in the detection of pathogens (e.g., various bacteria, viruses and eucarya) in clinical specimens, equipment, fixtures or products used to treat humans or animals as well as in clinical samples and clinical environments. For example, the analysis for microorganisms of interest can be performed using FISH or multiplex FISH using probes generated by the invention described herein (See: BP U.S. application Ser. Nos. 09/335,629 and 09/368,089).

The compositions, methods, kits, libraries and arrays of this invention are particularly useful in areas such as expression analysis, single nucleotide polymorphism (SNP) analysis, genetic analysis of humans, animals, fungi, yeast, viruses, and plants (including genetically modified organisms), therapy monitoring, pharmacogenomics, pharmacogenetics, epigenomics, and high throughput screening operations. The combinatorial libraries of this invention are useful for these probe intensive applications because they facilitate the massive, rapid, efficient and appropriately scaled synthesis of highly selective/discriminating combinatorial oligomers that do not have the disruption of duplex stability (i.e., the Tm penalty) caused by the chemistry that ligates the oligomer blocks as found in other types of combinatorial nucleobase oligomer libraries.

Multiplex Analysis

In certain embodiments, the invention provides insulating combinatorial nucleobase oligomers for use in multiplex hybridization assays. In a multiplex assay, numerous conditions of interest are simultaneously or sequentially examined. Multiplex analysis relies on the ability to sort sample components or the data associated therewith, during or after the assay is completed. In performing a multiplex assay, one or more distinct independently detectable moieties can be used to label two or more different insulating combinatorial nucleobase oligomers that are to be used simultaneously in an assay. As used herein, “independently detectable” means that it is possible to determine one label independently of, and in the presence of, at least one other additional label. The ability to differentiate between and/or quantitate each of the independently detectable moieties provides the means to multiplex a hybridization assay because the data correlates with the hybridization of each distinct, independently labeled insulating combinatorial nucleobase oligomer to a particular target sequence sought to be detected in the sample. Consequently, the multiplex assays of this invention can, for example, be used to simultaneously or sequentially detect the presence, absence, number, position or identity of two or more target sequences in the same sample in the same assay.

Blocking Probes

Blocking probes are nucleobase oligomers that can be used to suppress the binding of a second nucleobase sequence to a non-target sequence. In some embodiments, the second nucleobase oligomer is labeled. Preferred blocking probes are PNA probes (see, e.g., Coull et al., U.S. Pat. No. 6,110,676, herein incorporated by reference). The insulating combinatorial nucleobase oligomers of this invention can be used as blocking probes. Although these molecules are referred to as “probes” in the art, this is somewhat of a misnomer, as the nucleobase oligomer is not labeled nor otherwise detected.

Typically, blocking probes are closely related to the probing nucleobase sequence and preferably they comprise one or more single point mutations as compared with the target sequence sought to be detected in the assay. It is believed that blocking probes operate by hybridization to the non-target sequence to thereby form a more thermodynamically stable complex than is formed by hybridization between the probing nucleobase sequence and the non-target sequence. Formation of the more stable and preferred complex blocks formation of the less stable non-preferred complex between the probing nucleobase sequence and the non-target sequence, although an understanding of the mechanism is not required to make or use the invention. Thus, insulating combinatorial nucleobase oligomers of the invention find use as blocking probes to suppress the binding of a second nucleobase oligomer to a non-target sequence that might be present in an assay and thereby interfere with the performance of the assay (see, Fiandaca et al., “PNA Blocker Probes Enhance Specificity In Probe Assays”, Peptide Nucleic Acids: Protocols and Applications, pp. 129-141, Horizon Scientific Press, Wymondham, UK, 1999). The insulating combinatorial nucleobase oligomers of the invention also find use as the second (typically labeled) nucleobase molecule used in these protocols. The use of insulating combinatorial nucleobase oligomers of the invention as blocking probes extends more generally to use of the oligomers of the invention as any type of specific or non-specific nucleobase competitor in a hybridization reaction.

Polymerase Priming

The insulating combinatorial nucleobase oligomers of the invention find use in any application using primer extension, i.e., any reaction where an oligomer acts as a primer for template-dependent ribonucleotide (RNA) or deoxyribonucleotide (DNA) elongation by a polymerase enzyme. When the insulating combinatorial nucleobase oligomers are used as primers, they are required to have a structure that permits enzymatic elongation. This will typically require the presence of a minimum number of ribonucleotide or deoxyribonucleotide-containing subunits in the nucleobase oligomers in order to serve as a polymerase primer. In one embodiment, when the insulating combinatorial nucleobase oligomer is used as a primer, the oligomer is chimeric, such that the oligomer comprises nucleotides as well as other types of nucleobase structures, including for example, PNA.

It is noted that in one aspect, the expression “primer extension” has a specific meaning in reference to a molecular genetic technique for mapping a transcription start site. However, as used herein, the expression is used in its most general sense to describe any template-directed, primer-initiated polymerase reaction.

The art knows well the wide variety of applications utilizing primer extension reactions in experimental or diagnostic methodologies. Is not intended that the invention be limited in any way to the use of insulating combinatorial nucleobase oligomers in any particular type of primer extension reactions. Various primer extension reactions are widely used in modern molecular biology techniques. For example, Sanger nucleic acid sequencing utilizes a nucleobase oligomer primer annealed to a template, deoxyribonucleotide triphosphates (dNTPs), polymerase, and four dideoxynucleotide terminators that are combined in a reaction (the four teminators are either added to separate reactions or together in one reaction), and the reaction mixture is incubated under appropriate conditions to achieve primer extension.

In one aspect, the primer extension reaction is a polymerase chain reaction (PCR). The art knows well the protocols and diversity of applications that use PCR-based techniques. See, e.g., Mullis et al. (1986) Cold Spring Harbor Symposia on Quantitative Biology 51:263; Eckert et al. (1990) Nucl. Acids Res. 18:3739, Dieffenbach et al. (1995) PCR Primer: a laboratory manual, CSHL Press, Cold Springs Harbor, USA. Generally, a PCR reaction includes at least one template, at least one primer, at least one polymerase, and extendable nucleotides. At least one of the primers in the PCR reaction can be an insulating combinatorial nucleobase oligomer of the invention. The PCR reaction is subjected to temperature cycles that result in repeated annealing, primer extension and template dissociation in the reaction mix. This generates a primer extension product (or amplicon) complementary to at least a portion of the target template.

Analysis of microsatellites, including Variable Number of Tandem Repeats (VNTRs) and Short Tandem Repeats (STRs), is another widely used method employing primer extension reactions. STRs are sequences of two to seven nucleotides that are tandemly repeated at one or more locations in the genome. The number of tandem repeats varies from individual to individual. For certain genetic analysis techniques, STRs are amplified by PCR using specific primers flanking the repeat region and the number of repeats is determined. In certain techniques, the determination is made using size differentiation, e.g., by electrophoresis, mass spectroscopy, or chromatography.

Uses of Oligomer Blocks Comprising Universal Nucleobases

It is contemplated that the oligomer blocks comprising universal nucleobases find use in applications in addition to the synthesis of combinatorial oligomers. For example, an oligomer block comprising at least one universal nucleobase can be covalently attached (i.e., via a linker) to a solid phase via the chemically reactive moiety on the oligomer block and used as an affinity ligand for the isolation and/or purification of various molecules that bind to a particular nucleobase sequence. In one aspect, the solid phase can be, for example, a bead such as a SEPHAROSE® bead, and the bead can be immobilized in a chromatography column.

In one aspect, the molecule that binds to the nucleobases of the affixed oligomer block is another polynucleobase molecule that binds by the rules of base-pairing interactions. A nucleic acid (or other nucleobase-containing structure) can be isolated/purified following the formation of a hybridization complex between the nucleobases of the oligomer block that has been attached to a solid phase and a nucleobase target. This technique can find use in the analysis of fragmented or digested DNA following enzymatic degradation, for example, or in the analysis of DNA oligomers produced by expression or in other ways.

In another aspect, the oligomer block that is affixed to a solid phase can be used to isolate/purify proteins that bind to the nucleobases of the oligomer block in a sequence-specific manner. In this case, the nucleobases of the oligomer block can prepared in a single-stranded or double-stranded configuration for the isolation/purification of single-stranded binding proteins or double-stranded binding proteins.

Genomic Analysis

Insulating nucleobase oligomer blocks, insulating nucleobase oligomer block libraries, and insulating combinatorial nucleobase oligomers may be used in genomic analysis. For example, a target sample of genomic material may be contacted with a probe comprising insulating nucleobase oligomer blocks, or with insulating combinatorial nucleobase oligomers, in order to determine whether hybridization occurs. Hybridization between the probe and the target indicates the presence in the target of nucleobase sequence complementary to that of the probe.

In preferred embodiments of the invention, a target sample of genomic material may be contacted with a plurality of probes comprising insulating combinatorial nucleobase oligomers formed from an oligomer block library. Hybridization between one or more of the probes and the target indicates the presence in the target of nucleobase sequence complementary to that of the hybridizing probe sequence(s). Such hybridization may be detected by detecting fluorescence from fluorescent labels attached to the probes; by quenching of fluorescence from fluorescent labels attached to the probes; by anti-body binding to antigens on the probes; by detection of radioactivity emitted by radioactive labeled probes; or by other labeling and detection methods.

Gene Expression Analysis

Gene expression may be analyzed by detection of target gene or other nucleobase sequences in a sample indicative of gene expression, such as a cDNA derived from mRNA obtained from a cell of interest. For example, a cDNA library derived from a cell of interest may be contacted with a plurality of probes comprising insulating combinatorial nucleobase oligomers formed from an oligomer block library in order to detect the presence of nucleobase sequences complementary to those of the insulating combinatorial nucleobase oligomers. Such analysis may be used to determine the expression of particular nucelobase sequences and so be indicative of the expression of genes including such nucleobase sequences.

Such gene expression analysis may be performed on similar cells under different conditions or from cells during different parts of the cell cycle (see, for example, DeRisi et al., Science 278:680-686 (1997)). Comparison of the results of such gene expression analysis may be used to determine what gene activity is altered under the different conditions or during the different parts of the cell cycle. Similarly, comparison between normal cells and cancerous cells may indicate differences in gene expression between the normal and the cancerous conditions. Thus, for example, where cDNAs are obtained from normal and cancerous cells, comparison of the hybridization between such cDNAs and with insulating combinatorial nucleobase oligomers from an oligomer block library having featuresof the invention may be used to determine differences in gene expression between normal and cancerous cells.

### L. EXAMPLES

The following EXAMPLES are provided to further illustrate certain embodiments and aspects of the present invention. It is not intended that these EXAMPLES should limit the scope of any aspect of the invention. Although specific reaction conditions and reagents are described, it is clear that one familiar with the art would recognize alternative or equivalent conditions that also find use with the invention, where the alternative or equivalent conditions do not depart from the scope of the invention.

### Example 1

In this example, a combinatorial nucleobase oligomer is synthesized from two preformed oligomer blocks to form a combinatorial oligomer having a PNA structure.

Synthesis of a Combinatorial PNA oligomer from Two Presynthesized PNA Oligomer Blocks.

PNA oligomers are synthesized using commercially available reagents and instruments (Applied Biosystems) in scales ranging from submicromole to millimole scales. A, G, C and T PNA monomers are commercially available (Applied Biosystems). Universal base containing PNA monomers are synthesized by coupling of appropriately protected universal bases with Fmoc protected 2-aminoethyl glycine backbone via methylene carbonyl linker following published procedures (for example, Nielsen et al., Peptide Nucleic Acids; Protocols and Applications, Horizon Scientific Press, Norfolk, England, 1999). PNA synthesis utilizes standard peptide synthesis chemistry. Thus, both natural and non-natural amino acids and their derivatives are easily incorporated into PNA oligomers. Most conveniently PNA oligomer is synthesized by coupling Fmoc/Bhoc, tBoc/Z or MMT protected PNA monomers in the presence of ancillary reagents on an Expedite Synthesizer (Applied Biosystems) on derivatized XAL, PAL, PEG, PAM solid supports. A model 433A Peptide Synthesizer (Applied Biosystems) could also be used for PNA synthesis with MBHA support.

PNA is a polyamide and it has a C-terminus (carboxyl terminus) and an N-terminus (amino terminus) like peptides. The C-terminus is equivalent to 3′ end of oligonucleotides and N-terminus is equivalent 5′ end of oligonucleotides. The PNA oligomer will have a free amino group at the N-terminus and a carboxamide group at the C-terminus under standard synthesis and deprotection conditions. Synthesis of PNA with PAL, XAL and MBHA solid supports gives carboxamide at the C-terminus, and protected or free amino group at the N-terminus. However use of PEG and PAM solid supports will give free carboxyl group at the C-terminus and protected or free amino group at the N-terminus. The choice of solid support in PNA synthesis is determined by the intended use of PNA oligomer.

### Example 2

Synthesis of PNA Oligomer Probe Blocks for Human ApoE Gene.

| | | |
|---|---|---|
| Probe left half: | Reporter-CGGGXgly | (SEQ ID NO. 4) |
| Probe right half: | glyXCGCC-Quencher | (SEQ ID NO. 5) |

X is universal base and gly is glycine linker.

The left half of the probe requires a free carboxyl group at the C-terminus. This is provided by using glycine derivatized PEG support and G, C and universal base PNA monomer on a Expedite synthesizer in 2 micromole scale according to PNA synthesis protocol and manual provided by Applied Biosystems. The reporter dye is attached the N-terminus amino group while the PNA is attached to the solid support by standard procedures. Alternatively, the reporter dye may be attached to the N-terminus amnio group after the PNA has been cleaved from the support. Probe purification is by simple precipitation or by HPLC.

The right half of the probe is synthesized by using G, C, universal base PNA monomer, Fmoc protected glycine and PAL or XAL solid supports containing a quencher on a linker. The linker is lysine. In an alternative method, the quencher is attached to the side chain amino group of the lysine after cleavage of the PNA from the support. In this case the Fmoc protecting group of glycine unit is retained during the quencher coupling step. The probe is ready to couple with left half of the probe after removal of Fmoc group from glycine.

Synthesis of human ApoE gene probe from presynthesized left and right halves of the probe. The ligation of two halves is carried out in organic (ACN, DMF, NMP, DMSO etc.), aqueous (water or buffer) or a mixture of organic and aqueous solvents in presence of activator. The commonly used activators, such as EDC, HOAt, or a mixture of EDC and HOAT and other known activators are used in the coupling reaction. The probe halves are mixed in equimolar amounts with activator in appropriate solvent/s. The progress of the reaction is followed by HPLC. Purification of the product is accomplished by HPLC or by other methods. The probe is labeled by attachment of the fluorescent label 2′,7′dimethoxy-4′,5′-dichloro-6-carboxyrhodamine (JOE).

### Example 3

Use of a Combinatorial Oligomer Probe to Detect Specific Nucleotide Sequence in a Sample by Real-Time Monitoring

This EXAMPLE describes the monitoring of accumulation of PCR products corresponding to a segment of the Apolipoprotein E (ApoE) gene using a combinatorial oligomer probe in a TAQMAN® real-time PCR monitoring system. The samples being tested and compared are human genomic DNA samples isolated from immortalized cells lines (Coriell Cell Repository, Coriell Institute for Medical Research, Camden N.J.).

Twenty five microliter reactions are mixed, each containing:

| | |
|---|---|
| 12.5 | μl 2X TAQMAN ® Universal PCR Master Mix |
| | (Applied Biosystems) |
| 500 | nM ApoE forward primer: |
| | 5′ ACGCGGGCACGGCTGTC 3′ (SEQ ID NO: 6) |
| 500 | nM ApoE reverse primer: |
| | 5′ CTCGCGGATGGCGCTGA 3′ (SEQ ID NO: 7) |
| 200 | nM ApoE combinatorial probe: |
| | 5′ Reporter CGGCCNNCGCCGC-Quencher 3′, where |
| | N is universal base (SEQ ID NO: 8). |
| 10 | ng human genomic target DNA |

The ApoE segment is amplified by thermal cycling conditions that begin with 2 min at 50° C., 10 min at 95° C., and then 40 cycles of: 15 sec denaturation at 92° C. and 1 min annealing and extension at 60° C. Thermal cycling and real-time fluoprescence detection is conducted on an ABI PRISM® 7700 Sequence Detection System (Applied Biosystems).

Detection of reporter signal above the fluorescence background indicates the presence of the ApoE segment in the human genomic DNA sample.

### Example 4

Use of a Combinatorial Oligomer Probe Library to Detect Specific Nucleotide Sequences in a Sample

This EXAMPLE describes the monitoring of the occurrence of nucleobase sequences during the production of ApoE PCR products using a library of insulating combinatorial nucleobase oligomers as probes. The samples being tested and compared are human genomic DNA samples isolated from immortalized cells lines (Coriell Cell Repository, Coriell Institute for Medical Research, Camden N.J.). Different insulating combinatorial nucleobase oligomers are added to each well in a series of 96-well plates. In this example, 4096 insulating combinatorial nucleobase oligomers formed from the linkage of oligomer blocks having three specifity-determining nucleobases and one universal base (of a possible 65,536 insulating combinatorial nucleobase oligomers comprising a complete library of such probes) are added to a corresponding number of wells in three 1,536-well plates (Nalge Nunc, Rochester N.Y. 14625 USA).

Twenty five microliter reactions are mixed, each containing:

| | |
|---|---|
| 12.5 | μl 2X TAQMAN ® Universal PCR Master Mix |
| | (Applied Biosystems) |
| 500 | nM ApoE forward primer: |
| | 5′ ACGCGGGCACGGCTGTC 3′ (SEQ ID NO: 6) |
| 500 | nM ApoE reverse primer: |
| | 5′ CTCGCGGATGGCGCTGA 3′ (SEQ ID NO: 7) |
| 200 | nM insulating combinatorial nucleobase |
| | oligomer probe. |
| 10 | ng human genomic target DNA |

The ApoE segment is amplified by thermal cycling conditions that begin with 2 min at 50° C., 10 min at 95° C., and then 40 cycles of: 15 sec denaturation at 92° C. and 1 min annealing and extension at 60° C. Thermal cycling and real-time fluoprescence detection is conducted on an ABI PRISM® 7700 Sequence Detection System (Applied Biosystems).

Detection of reporter signal in a well above the fluorescence background indicates the hybridization of the insulating combinatorial nucelobase oligomer added to that well with the ApoE segment in the human genomic DNA sample.

### Example 5

Use of a Combinatorial Oligomer Probe Library Array to Detect Specific Nucleotide Sequences in a Sample

This EXAMPLE describes the hybridization of nucleobase sequences with ApoE PCR products using a library of insulating combinatorial nucleobase oligomers affixed to an array as probes of hybridization. The samples being tested and compared are human genomic DNA samples isolated from immortalized cells lines (Coriell Cell Repository, Coriell Institute for Medical Research, Camden N.J.).

Microarrays together containing all possible 65,536 insulating combinatorial nucleobase oligomers formed from the linkage of oligomer blocks having three specifity-determining nucleobases and one universal base (comprising a complete library of such probes) are produced by spotting solutions containing the oligomers onto glass substrates (DeRisi et al., Science 278:680-686 (1997); Lashkari et al., P.N.A.S. 94:13057-13062 (1997)).

The human genomic DNA sample is prepared as described above. Purified cDNA is resuspended in 11 μl of 3.5× sodium chloride-sodium citrate (SSC) containing 10 μg poly(dA) and 0.3 μl of 10% sodium dodecyl sulfate (SDS). The solution is boiled for two minutes and then allowed to cool to room temperature before being applied to the microarray plates under a cover slip. The slides are then placed in a hybridization chamber and incubated in a water bath at 62° C. for 10 hours. Slides are then washed in 2×SSC, 0.2% SDS for 5 min, then washed in 0.05×SSC for 1 min. Slides are then dried by centrifugation at 500 rpm in a Beckman CS-6R centrifuge.

Slides are scanned in a GenePix 4000 microarray scanner Axon Instruments, UnionCity, Calif. 94587 USA). Detection of reporter signal at a location on the array above the fluorescence background indicates the hybridization of the insulating combinatorial nucelobase oligomer at that location with the ApoE segment in the human genomic DNA sample.

All patents, published patent applications and publications mentioned in the above specification are herein incorporated by reference in their entirety. Various modifications and variations of the described compositions and methods of the invention will be apparent to those skilled in the art without departing from the scope and spirit of the invention. Although the invention has been described in connection with various specific embodiments, it should be understood that the invention as claimed should not be unduly limited to such specific embodiments. Indeed, various modifications of the described modes for carrying out the invention which are obvious to those skilled in the art are intended to be within the scope of the following claims.

1. An insulating nucleobase oligomer block library comprising a plurality of insulating nucleobase oligomer blocks, wherein
each oligomer block independently comprises a sequence of polymerized nucleobases having termini, said sequence including at least three specificity-determining nucleobases and at least one universal nucleobase, and at least one chemically reactive moiety that is covalently coupled to a terminus of the polymerized nucleobase sequence, wherein a universal nucleobase comprises a base that does not significantly discriminate between bases on a complementary polymeric structure having nucleobases, and a specificity-determining nucleobase is capable of discriminating between bases on a complementary polymeric structure having nucleobases,
the chemically reactive moiety on one oligomer block is capable of reacting with the chemically reactive moiety on at least one other oligomer block to form a covalent linker between the oligomer blocks in the absence of a template to form an insulating combinatorial nucleobase oligomer, and
the insulating combinatorial nucleobase oligomer has a hybridization target sequence that is a composite of the specificity-determining nucleobases in the oligomer blocks comprising the insulating combinatorial nucleobase oligomer.

2. The insulating nucleobase oligomer block library of claim 1, wherein each oligomer block independently comprises from about 1 to about 10 universal nucleobases.

3. The insulating nucleobase oligomer block library of claim 2, wherein each oligomer block independently comprises from about 1 to about 3 universal nucleobases.

4. The insulating nucleobase oligomer block library of claim 1, wherein each oligomer block independently comprises from about 3 to about 8 specificity-determining nucleobases.

5. The insulating nucleobase oligomer block library of claim 1, wherein said universal nucleobase is proximal to said chemically reactive moiety.

6. The insulating nucleobase oligomer block library of claim 1, wherein said universal nucleobase is adjacent to said chemically reactive moiety.

7. The insulating nucleobase oligomer block library of claim 1, wherein said universal nucleobase is distal to the chemically reactive moiety.

8. The insulating nucleobase oligomer block library of claim 1, wherein said universal nucleobase is between and adjacent to two specificity-determining nucleobases.

9. The insulating nucleobase oligomer block library of claim 1, wherein said universal nucleobase comprises a universal base selected from hypoxanthine, 5-nitro,1-(β-D-2-deoxyribofuranosyl)indole (termed 5-nitroindole), 1-(2′-deoxy-β-D-ribofuranosyl)-3-nitropyrrole (termed 3-nitropyrrole), 7-azaindole (7AIT), N8-(7-deaza-8aza-adenine), (B) 6-methyl-7-azaindole (M7AI), (C) pyrrollpyrizine (PP), (D) imidizopyridine (ImPy), (E) isocarbostyril (ICS), (F) propynyl-7-azaindole (P7AI), (G) propynylisocarbostyril (PICS), (H) allenyl-7-azaindole (A7AI) and N8-(7-deaza-8-aza-adenine).

10. The insulating nucleobase oligomer block library of claim 1, wherein said first and said second chemically reactive moieties are selected from carboxyl groups, ketones, aldehydes, dienes, dienophiles, hydrazines, semicarbazides, amino groups, aminoxy groups, halides, and sulfhydryl groups.

11. The insulating nucleobase oligomer block library of claim 1, wherein said first chemically reactive moiety comprises a carboxyl group, and said second chemically reactive moiety comprises an amino group.

12. The insulating nucleobase oligomer block library of claim 1, wherein said first chemically reactive moiety comprises a diene, and said second chemically reactive moiety comprises a dienophile.

13. The insulating nucleobase oligomer block library of claim 1, wherein said first chemically reactive moiety comprises a hydrazine, and said second chemically reactive moiety comprises a semi-carbazide.

14. The insulating nucleobase oligomer block library of claim 1, wherein each oligomer block independently comprises nucleobases that are peptide nucleic acid (PNA), D-deoxyribonucleotides, L-deoxyribonucleotides, locked nucleic acid (LNA), 2′-O-alkyl oligonucleotides, 3′ modified oligodeoxyribonucleotides, N3′-P5′ phosphoramidate (NP) oligomers, MGB-oligonucleotides, phosphorothioate (PS) oligomers, C₁-C₄ alkylphosphonate oligomers, phosphoramidates, β-phosphodiester oligonucleotides, or α-phosphodiester oligonucleotides.

15. The insulating nucleobase oligomer block library of claim 1, wherein an oligomer block comprises nucleobases of two or more different structures, and that oligomer block is chimeric.

16. The insulating nucleobase oligomer block library of claim 15, wherein said nucleobases of the chimeric oligomer block comprise at least two structures selected from peptide nucleic acid (PNA), D-deoxyribonucleotides, L-deoxyribonucleotides, locked nucleic acid (LNA), 2′-O-alkyl oligonucleotides, 3′ modified oligodeoxyribonucleotides, N3′-P5′ phosphoramidate (NP) oligomers, MGB-oligonucleotides, phosphorothioate (PS) oligomers, C₁-C₄ alkylphosphonate oligomers, phosphoramidates, β-phosphodiester oligonucleotides, or α-phosphodiester oligonucleotides.

17. The insulating nucleobase oligomer block library of claim 1, wherein said oligomer block comprises one or more protecting groups.

18. The insulating nucleobase oligomer block library of claim 1, wherein said oligomer block comprises at least one label capable of providing a detectable signal.

19. The insulating nucleobase oligomer block library of claim 18, wherein said label is selected from the group of labels consisting of dyes, fluorescent labels, luminescent labels, radioactive labels, antigens, haptens, enzymes, enzyme substrates, protecting groups, and chemically reactive groups.

20. The insulating nucleobase oligomer block library of claim 18, wherein said label is capable of interacting with a second label.

21. The insulating nucleobase oligomer block library of claim 19, wherein said hapten is selected from the group consisting of fluorescein, biotin, 2,4-dinitrophenyl, digoxigenin, lipopolysaccharide; apotransferrin; ferrotransferrin; insulin; a cytokine; gp120; β-actin; leukocyte function-associated antigen 1 (LFA-1; CD11a/CD18j; Mac-1 (CD11b/CD18); glycophorin; laminin; collagen; fibronectin; vitronectin; an integrin, ankyrin; fibrinogen, Factor X; inter-cellular adhesion molecule 1 (ICAM-1); inter-cellular adhesion molecule 2 (ICAM-2); spectrin, fodrin; CD4; a cytokine receptor; an insulin receptor; a transferrin receptor; Fe; polymyxin B; endotoxin-neutralizing protein (ENP); an antibody-specific antigen; avidin; streptavidin; and biotin.

22. The insulating nucleobase oligomer block library of claim 1, said library comprising at least 64 different insulating nucleobase oligomer blocks having different sequences of specificity-determining nucleobases.

23. The insulating nucleobase oligomer block library of claim 1, said sequence including at least four specificity-determining nucleobases, said library comprising at least 256 different insulating nucleobase oligomer blocks having different sequences of specificity-determining nucleobases.

24. The insulating nucleobase oligomer block library of claim 1, said sequence including at least five specificity-determining nucleobases, said library comprising at least 1024 different insulating nucleobase oligomer blocks having different sequences of specificity-determining nucleobases.

25. The insulating nucleobase oligomer block library of claim 1, said sequence including at least six specificity-determining nucleobases, said library comprising at least 4096 different insulating nucleobase oligomer blocks having different sequences of specificity-determining nucleobases.

26. An insulating combinatorial nucleobase oligomer, comprising a plurality of covalently linked insulating nucleobase oligomer blocks, said plurality of covalently linked insulating nucleobase oligomer blocks comprising at least a first insulating nucleobase oligomer block covalently linked to a second insulating nucleobase oligomer block by a covalent linker, wherein:
said first and said second insulating nucleobase oligomer blocks each comprise at least three specificity-determining nucleobases covalently linked to a polymeric backbone structure, and at least one universal nucleobase covalently linked to said backbone structure; and
said covalent linker comprises a chemical bond resulting from a chemical reaction between chemically reactive moieties of said first and said second insulating nucleobase oligomer blocks in the absence of a template.

27. The insulating combinatorial nucleobase oligomer of claim 26, comprising a dimer of said insulating nucleobase oligomer block oligomers.

28. The insulating combinatorial nucleobase oligomer of claim 26, wherein a universal nucleobase is adjacent to said chemically reactive moiety.

29. The insulating combinatorial nucleobase oligomer of claim 26, wherein a universal nucleobase is distal to the chemically reactive moiety.

30. The insulating combinatorial nucleobase oligomer of claim 26, wherein a universal nucleobase is between and adjacent to two specificity-determining nucleobases.

31. The insulating combinatorial nucleobase oligomer of claim 26, wherein a universal nucleobase comprises a universal base selected from hypoxanthine, 5-nitro,1-(β-D-2-deoxyribofuranosyl)indole (termed 5-nitroindole), 1-(2′-deoxy-β-D-ribofuranosyl)-3-nitropyrrole (termed 3-nitropyrrole), 7-azaindole (7AIT), N8-(7-deaza-8aza-adenine), (B) 6-methyl-7-azaindole (M7AI), (C) pyrrollpyrizine (PP), (D) imidizopyridine (ImPy), (E) isocarbostyril (ICS), (F) propynyl-7-azaindole (P7AI), (G) propynylisocarbostyril (PICS), (H) allenyl-7-azaindole (A7AI) and N8-(7-deaza-8-aza-adenine).

32. The insulating combinatorial nucleobase oligomer of claim 26, wherein said covalent linker is selected from an amide linkage, a peptide linkage, an amino linkage, an aminoxy linkage, a diene, a semicarbazone, and a sulfide linkage.

33. The insulating combinatorial nucleobase oligomer of claim 32, wherein said linker is an amino acid linker.

34. The insulating combinatorial nucleobase oligomer of claim 33, wherein said linker is selected from a glycine (gly) linker, a lysine (lys) linker, a glutamic acid (glu) linker, a cysteine (cys) linker, an aspartic acid (asp) linker, and an ornithine linker.

35. The insulating combinatorial nucleobase oligomer of claim 33, wherein said linker is a two-amino acid linker.

36. The insulating combinatorial nucleobase oligomer of claim 35, wherein said two-amino acid linker is selected from glycine-glycine, lysine-glycine, glutamic acid-glycine, glycine-cysteine, cysteine-glycine, aspartic acid-glycine, aspartic acid-glutamic acid, arginine-glycine, lysine-X, arginine-X, glutamic acid-X, aspartic acid-X, asparagine-X, phenylalanine-X, leucine-X, and ornithine-X, where X represents any naturally-occurring amino acid.

37. The insulating combinatorial nucleobase oligomer of claim 26, wherein said first and said second insulating nucleobase oligomer blocks comprise from about three to about eight specificity-determining nucleobases.

38. The insulating combinatorial nucleobase oligomer of claim 26, wherein said first and said second insulating nucleobase oligomer blocks comprise from about one to about ten universal nucleobases.

39. The insulating combinatorial nucleobase oligomer of claim 38, wherein said first and said second insulating nucleobase oligomer blocks comprise from about one to about three universal nucleobases.

40. The insulating combinatorial nucleobase oligomer of claim 26, wherein said covalent linker is adjacent a universal nucleobase.

41. The insulating combinatorial nucleobase oligomer of claim 26, wherein said covalent linker is adjacent said a specificity-determining nucleobase.

42. The insulating combinatorial nucleobase oligomer of claim 26, wherein said polymeric backbone structure forms part of a sequence of peptide nucleic acid (PNA), D-deoxyribonucleotides, L-deoxyribonucleotides, locked nucleic acid (LNA), 2′-O-alkyl oligonucleotides, 3′ modified oligodeoxyribonucleotides, N3′-P5′ phosphoramidate (NP) oligomers, MGB-oligonucleotides, phosphorothioate (PS) oligomers, C₁-C₄ alkylphosphonate oligomers, phosphoramidates, β-phosphodiester oligonucleotides, or α-phosphodiester oligonucleotides.

43. The insulating combinatorial nucleobase oligomer of claim 26, comprising at least one label capable of providing a detectable signal.

44. The insulating combinatorial nucleobase oligomer of claim 43, wherein said label is selected from the group of labels consisting of dyes, fluorescent labels, luminescent labels, radioactive labels, antigens, haptens, enzymes, enzyme substrates, protecting groups, and chemically reactive groups.

45. The insulating combinatorial nucleobase oligomer of claim 43, wherein said label is capable of interacting with a second label.

46. The insulating combinatorial nucleobase oligomer of claim 44, wherein said hapten is selected from the group consisting of fluorescein, biotin, 2,4-dinitrophenyl, digoxigenin, lipopolysaccharide; apotransferrin; ferrotransferrin; insulin; a cytokine; gp120; β-actin; leukocyte function-associated antigen 1 (LFA-1; CD11a/CD18); Mac-1 (CD11b/CD18); glycophorin; laminin; collagen; fibronectin; vitronectin; an integrin, ankyrin; fibrinogen, Factor X; inter-cellular adhesion molecule 1 (ICAM-1); inter-cellular adhesion molecule 2 (ICAM-2); spectrin, fodrin; CD4; a cytokine receptor; an insulin receptor; a transferrin receptor; Fe⁺⁺⁺; polymyxin B; endotoxin-neutralizing protein (ENP); an antibody-specific antigen; avidin; streptavidin; and biotin.

47. A method for the synthesis of an insulating combinatorial nucleobase oligomer, comprising:
selecting two or more oligomer blocks from the oligomer block library of claim 0, where the chemically reactive moieties on the oligomer blocks are capable of reacting to form a covalent linker between the oligomer blocks in the absence of a template, and
reacting the selected oligomer blocks under suitable conditions whereby the chemically reactive moieties on the oligomer blocks combine to form a covalent linker between the oligomer blocks, thereby forming the insulating combinatorial nucleobase oligomer.

48. The method of claim 47, wherein said insulating combinatorial nucleobase oligomer is enzymatically non-extendable.

49. The method of claim 48, wherein said insulating combinatorial nucleobase oligomer comprises modified nucleotides or internucleotide analogs.

50. The method of claim 48, wherein said insulating combinatorial nucleobase oligomer comprises peptide nucleic acid.

51. The method of claim 47, wherein the nucleobases comprising the oligomer blocks are of different structures, and the insulating combinatorial nucleobase oligomer is chimeric.

52. The method of claim 47, wherein said insulating combinatorial nucleobase oligomer is enzymatically extendable by at least one polymerase enzyme.

53. The method of claim 47, wherein said insulating combinatorial nucleobase oligomer further comprises one or more label.

54. The method of claim 53, wherein said label is connected at an oligomer block terminus, at a position internal to an oligomer block or at a position integral to the linker.

55. The method of claim 53, wherein said label is selected from the group consisting of a chromophore, a fluorochrome, a fluorophore, a quencher, a spin label, a radioisotope, an enzyme, a hapten, a chemiluminescent compound and a bioluminescent compound.

56. The method of claim 53, wherein said insulating combinatorial nucleobase oligomer comprises at least one energy transfer set of labels, where the set of labels comprises at least one acceptor moiety and at least one donor moiety.

57. The method of claim 56, wherein the labels of the energy transfer set are linked to the termini of the insulating combinatorial nucleobase oligomer or to sites within the insulating combinatorial nucleobase oligomer.

58. The method of claim 56, wherein the energy transfer set comprises a single donor moiety and a single acceptor moiety.

59. The method of claim 56, wherein said insulating combinatorial nucleobase oligomer is a probe for real-time PCR monitoring.

60. The method of claim 56, wherein the energy transfer set of labels are linked to the insulating combinatorial nucleobase oligomer at positions that facilitate a change in detectable signal in at least one of the labels when the insulating combinatorial oligomer is hybridized to a target sequence as compared to when the insulating combinatorial nucleobase oligomer is not hybridized to a target sequence.

61. The method of claim 56, wherein said acceptor moiety and donor moiety are coupled to different oligomer blocks.

62. The method of claim 58, wherein both the acceptor and donor moieties are fluorophores.

63. The method of claim 58, wherein the donor moiety is a donor fluorophore and the acceptor is a non-fluorescent quencher moiety.

64. The method of claim 55, wherein said enzyme is selected from the group consisting of alkaline phosphatase, soybean peroxidase, horseradish peroxidase, ribonuclease, urease, glucoamylase, β-galactosidase and protease.

65. The method of claim 55, wherein said hapten is selected from the group consisting of fluorescein, biotin, 2,4-dinitrophenyl, digoxigenin, lipopolysaccharide; apotransferrin; ferrotransferrin; insulin; a cytokine; gp120; β-actin; leukocyte function-associated antigen 1 (LFA-1; CD11a/CD18); Mac-1 (CD11b/CD18); glycophorin; laminin; collagen; fibronectin; vitronectin; an integrin, ankyrin; fibrinogen, Factor X; inter-cellular adhesion molecule 1 (ICAM-1); inter-cellular adhesion molecule 2 (ICAM-2); spectrin, fodrin; CD4; a cytokine receptor; an insulin receptor; a transferrin receptor; Fe⁺⁺⁺; polymyxin B; endotoxin-neutralizing protein (ENP); an antibody-specific antigen; avidin; streptavidin; and biotin.

66. The method of claim 47, wherein said insulating combinatorial nucleobase oligomer is affixed to a solid support.

67. The method of claim 66, wherein said solid support comprises a material selected from silica, reverse-phase silica, organic polymers, oligosaccharides, nitrocellulose, diazocellulose, glass, controlled-pore-glass (CPG), polystyrene, polyvinylchloride, polypropylene, polyethylene, polyfluoroethylene, polyethyleneoxy, polyacrylamide, co-polymers and grafts of polymers.

68. The method of claim 66, where said insulating combinatorial nucleobase oligomer exists in an array comprising more than one insulating combinatorial nucleobase oligomer.

69. A complex comprising a target and an insulating combinatorial nucleobase oligomer produced by the method of claim 47, wherein
said target has a nucleobase sequence that is complementary to the specificity-determining nucleobases of the oligomer blocks that are joined to produce the insulating combinatorial nucleobase oligomer, and
said complex is formed by hybridization of said insulating combinatorial nucleobase oligomer with said target such that base pairing occurs between the nucleobases of the target and the universal and specificity-determining nucleobases of the oligomer.

70. The complex of claim 69 where the specificity-determining nucleobases within the insulating combinatorial nucleobase oligomer bind to a contiguous target sequence.

71. The complex of claim 69 where the sequence of specificity-determining nucleobases within the insulating combinatorial nucleobase oligomer binds to a noncontiguous target sequence.

72. The complex of claim 69 where the sequence of specificity-determining nucleobases within the insulating combinatorial nucleobase oligomer binds to a gapped target sequence.

73. An insulating nucleobase oligomer block construct, comprising:
A solid support; and
An insulating nucleobase oligomer block comprising a sequence of polymerized nucleobases having a terminus, said sequence including at least three specificity-determining nucleobases and at least one universal nucleobase, and at least one chemically reactive moiety that is covalently coupled to a terminus of the polymerized nucleobase sequence,
Wherein the insulating combinatorial nucleobase oligomer has a hybridization target sequence that is a composite of the specificity-determining nucleobases in the oligomer blocks comprising the insulating combinatorial nucleobase oligomer, and
the chemically reactive moiety is capable of reacting with a chemically reactive moiety on another oligomer block to form a covalent linker between the oligomer blocks in the absence of a template to form an insulating combinatorial nucleobase oligomer affixed to said solid support.

74. The insulating nucleobase oligomer block construct of claim 73, wherein said solid support comprises a solid support selected from silica, reverse-phase silica, organic polymers, oligosaccharides, nitrocellulose, diazocellulose, glass, controlled-pore-glass (CPG), polystyrene, polyvinylchloride, polypropylene, polyethylene, polyfluoroethylene, polyethyleneoxy, polyacrylamide, co-polymers and grafts of polymers, dextran, agar, agarose, SEPHAROSE®, SEPHADEX® SEPHACRYL®, cellulose, starch, nylon, latex beads, magnetic beads, paramagnetic beads, superparamagnetic beads, and microtitre plates.

75. The insulating nucleobase oligomer block construct of claim 73, comprising a plurality of different insulating combinatorial nucleobase oligomers having different sequences of specificity-determining nucleobases.

76. The insulating nucleobase oligomer block construct of claim 75, wherein said solid support comprises an array of different insulating combinatorial nucleobase oligomers having different sequences of specificity-determining nucleobases.
